SEQUENCE LISTING (1 sequence)

SEQ ID NO: 1 - protein, 5 aa, Artificial Sequence
  cyclic peptide
RGDFK

(19) United States
(12) Patent Application Publication
(10) Pub. No.: US 2022/0409743 A1
(43) Pub. Date: Dec. 29, 2022

(54) VIRUS-MIMETIC NANOPARTICLES

(71) Applicant: UNIVERSITÄT REGENSBURG, REGENSBURG (DE)

(72) Inventors: SARA MALSANKA-FIGUEROA, NÜRNBERG (DE); DANIEL FLEISCHMANN, REGENSBURG (DE); ACHIM GÖPFERICH, SINZING (DE)

(21) Appl. No.: 17/781,112

(22) PCT Filed: Dec. 9, 2020
(86) PCT No.: PCT/EP2020/085363

§ 371 Date: May 31, 2022

(30) Foreign Application Priority Data
Dec. 23, 2019 (EP) 19219424.9

(51) Int. Cl. A61K 47/69; A61K 31/197; A61K 31/4418; A61K 47/54; A61K 47/59; A61K 47/60; A61K 47/62; A61P 27/02; A61P 35/00; A61P 13/12; C07K 16/18

(52) U.S. Cl. CPC A61K 47/6937; A61K 31/197; A61K 31/4418; A61K 47/54; A61K 47/593; A61K 47/60; A61K 47/62; A61P 27/02; A61P 35/00; A61P 13/12; C07K 16/18

(57) ABSTRACT

The present invention relates to a nanoparticle comprising a nanomaterial and at least a first ligand and a second ligand tethered to the nanoparticle. The present invention further relates to a nanoparticle for use as a medicament or diagnostic agent. The present invention also relates to a nanoparticle for use in a method of preventing or treating a disease selected from diabetic nephropathy, glomerulonephritis, glomerular VEGF A dysregulation, endothelial VEGF A dysregulation, diabetic retinopathy, rheumatoid arthritis, age-related macular degeneration, and cancer such as breast cancer. Furthermore, the present invention relates to a method of preparing a nanoparticle.

### FIELD OF THE INVENTION

The present invention relates to a nanoparticle comprising a nanomaterial and at least a first ligand and a second ligand. The present invention further relates to a nanoparticle for use as a medicament or diagnostic agent. The present invention also relates to a nanoparticle for use in a method of preventing or treating a disease selected from diabetic nephropathy, glomerulonephritis, glomerular VEGF A dysregulation, endothelial VEGF A dysregulation, diabetic retinopathy, rheumatoid arthritis, age-related macular degeneration, and cancer such as breast cancer. Furthermore, the present invention relates to a method of preparing a nanoparticle.

### BACKGROUND OF THE INVENTION

Myriads of nanomaterials have been developed over recent years as carriers for drug therapy or diagnostics. To outfit them with the ability to identify target cells with sufficient specificity in vivo, ligands that bind to cellular receptors have been tethered to their surfaces. However, simply following this old paradigm increased the avidities of nanomaterials but turned out to be insufficient for unequivocal cell identification. Presently, not even nanomaterials that present several different ligands for hetero-multivalent binding are able to distinguish between different cells types.

Viruses in contrast, are nanoparticles (NPs) with ultimate target cell specificity. In contrast to synthetic biomedical nanomaterials, viruses make use of a consecutive multistep recognition process for cell identification. As many current nanoparticle-based approaches lack sufficient specificity, exploitation of viral targeting strategies could be a viable option to overcome this limitation. Thus, mimicking the sequential recognition strategy of a virus, such as influenza A virus, with nanomaterials might allow to specifically target cells. Particularly, a viral target cell recognition is likely advantageous in vivo, where particles are subject to surface modifications due to protein adsorption.

Especially the initial step of viral attachment to cell membranes, which does not result in particle uptake but increases virus particle density on the cell surface, is missing in present nanoparticle design strategies. This initial adhesion to glycolipids and glycoproteins or specific receptors was found to be essential for viral infectivity. More so, if particles are subjected to clearance once they reach the target tissue, they are rendered inadequate for drug delivery purposes. In the case of the mesangium early reports have shown that small particles having a diameter of 70±25 nm or less were penetrating the glomerular endothelial fenestrations having a diameter of about 80-100 nm. However, these particles have been subject to mesangial clearance. This problem of known nanoparticles often being rapidly cleared from the tissue could be overcome if nanoparticles are internalized by target cells in a target tissue.

Maslanka Figueroa et al. [1] relate to polymer nanoparticles comprising a ligand which is angiotensin-I.

Sah et al. [2] relate to nanoparticles comprising block-copolymers and a drug.

The present invention aims at providing nanomaterials outfitted with virus-mimetic cell identification mechanisms for addressing cells in vitro and in vivo. Furthermore, an aim of the present invention is to provide nanoparticles that allow for an effective accumulation of said nanoparticles in a target tissue in vivo. A further aim of the present invention is providing a drug delivery system that allows to deliver a drug or a diagnostic agent to a target tissue.

### SUMMARY OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In a first aspect, the present invention relates to a nanoparticle, comprising a nanomaterial and at least a first ligand and a second ligand,
wherein said first ligand is capable of mediating an attachment of said nanoparticle to a target cell, and
wherein said second ligand is capable of mediating an internalization of said nanoparticle into said target cell, and
wherein, preferably, said nanomaterial comprises any of polyethylene glycol (PEG), polylactic acid (PLA), poly(lactic-co-glycolic acid) (PLGA), an oxazoline-derived polymer, a poly(aminoacid), a polysaccharide, a phospholipid, a sphingolipid, cholesterol, a PEG-lipid, a block-copolymer such as PEG-PLA or PEG-poly-caprolactone, an inorganic substance such as gold or a qdot material, and a combination thereof.

In one embodiment, said first ligand is a non-agonistic agent binding to a GPCR, such as angiotensin II receptor type 1 (AT1r), human neuropeptide Y1-receptor, and C-X-C chemokine receptor type 4, and/or an agent binding to glycoprotein and/or glycolipid on a target cell surface, such as a heparan sulfate, a sialoglycoprotein, a ganglioside, and a mannose receptor, preferably is EXP3174 or telmisartan.

In one embodiment, said second ligand is any of i) an agent binding to an integrin, such as αVβ3 integrin or αVβ5 integrin, preferably selected from RGD, a cyclic RGD-peptide having a sequence of SEQ ID NO. 1, and derivatives thereof, ii) an agonistic agent binding to a GPCR such as AT1r, preferably activated angiotensin-II, iii) an agent binding to an ectoenzyme, such as legumain, a membrane-type matrix metalloproteinase, and angiotensin converting enzyme (ACE), preferably angiotensin-I, and/or iv) an agent binding to a transferrin-receptor.

In one embodiment, said nanoparticle further comprises a therapeutic agent, preferably any of pirfenidone and cinaciguat.

In one embodiment, said first ligand and said second ligand are each coupled to said nanomaterial, preferably are each coupled to a block-copolymer chain of said nanomaterial.

In one embodiment, said nanomaterial comprises more than one block-copolymer chain, and said first ligand is coupled to a first block-copolymer chain of said nanomaterial and said second ligand is coupled to a second block-copolymer chain of said nanomaterial, and said first block-copolymer chain is longer than said second block-copolymer chain, preferably at least 1.5× longer than said second block-copolymer chain, more preferably at least 3× longer than said second block-copolymer chain.

In one embodiment, said first block-copolymer chain comprises PEG in a range of from 1 k to 20 k, preferably 1 k to 10 k, and/or comprises PLA in a range of from 5 k to 40 k, preferably 10 k to 20 k, optionally said first block-copolymer chain is PEG₅ₖ-PLA₁₀ₖ and said second block-copolymer chain is PEG₂ₖ-PLA₁₀ₖ.

In one embodiment, said second ligand is enzymatically activated prior to said internalization of said nanoparticle into said target cell.

In one embodiment, said target cell is selected from a mesangial cell, an endothelial cell, such as a retinal endothelial cell, a B cell, a T cell, a macrophage, a dendritic cell, and a tumor cell.

In one embodiment, said particle has a size of from 5 nm to 1000 nm, preferably of from 10 nm to 150 nm, more preferably of from 20 nm to 100 nm.

In one embodiment, a ratio of said first ligand to said second ligand is in the range of from 2:1 to 1:2, preferably is 1:1.

In one embodiment, said particle has a particle avidity for a targeted receptor of from 1 pM to 100 nM, preferably 5 pM to 1 nM.

In one embodiment, said nanomaterial comprises PEG and said particle has a ligand density of ligand/PEG of at least 5%, preferably of at least 15%, more preferably of at least 25%.

In a further aspect, the present invention relates to a nanoparticle, as defined in any of the embodiments above, for use as a medicament or diagnostic agent.

In a further aspect, the present invention relates to a nanoparticle, as defined in any of the embodiments above, for use in a method of preventing or treating a disease selected from diabetic nephropathy, glomerulonephritis, glomerular VEGF A dysregulation, endothelial VEGF A dysregulation, diabetic retinopathy, rheumatoid arthritis, age-related macular degeneration, and cancer such as breast cancer.

In a further aspect, the present invention relates to a method of preparing a nanoparticle comprising the steps:
a) Providing, in any order, one or several nanomaterial(s), preferably comprising any of polyethylene glycol (PEG), polylactic acid (PLA), poly(lactic-co-glycolic acid) (PLGA), an oxazoline-derived polymer, a poly(aminoacid), a polysaccharide, a phospholipid, a sphingolipid, cholesterol, a PEG-lipid, a block-copolymer such as PEG-PLA or PEG-poly-caprolactone, an inorganic substance such as gold or a qdot material, and a combination thereof, and, optionally, a therapeutic agent;
b) optionally, preparing a block-copolymer from any of said one or several nanomaterial(s);
c) coupling, in one or more steps, a first ligand and a second ligand thereto, preferably by means of DCC/NHS- or EDC/NHS-coupling;
d) providing a therapeutic agent, preferably a lipophilic therapeutic agent, if not already provided in step a);
e) preparing and obtaining a nanoparticle using the ligands coupled to said nanomaterial and said therapeutic agent, preferably by nanoprecipitation.

In one embodiment, said obtaining in step e) comprises obtaining nanoparticles having a polydispersity index of from 0.01 to 0.5, preferably from 0.01 to 0.3, more preferably from 0.01 to 0.1.

In this aspect, said nanoparticle, said nanomaterial, said therapeutic agent, said block-copolymer, said first ligand, and said second ligand are as defined above.

In a further aspect, the present invention relates to a pharmaceutical composition comprising a nanoparticle, as defined above, and a pharmaceutically acceptable excipient.

In a further aspect, the present invention relates to a method of preventing or treating a disease, wherein said method comprises administering an effective amount of a nanoparticle and/or a pharmaceutical composition to a patient in need thereof.

In this aspect, said disease, said nanoparticle, said pharmaceutical composition are as defined above.

In a further aspect, the present invention relates to the use of a nanoparticle in the manufacture of a medicament for the treatment of a disease.

In this aspect, said nanoparticle and said disease are as defined above.

In a further aspect, the present invention relates to the use of a nanoparticle in the manufacture of a diagnostic agent for the diagnosis and/or prognosis of a disease.

In this aspect, said nanoparticle and said disease are as defined above.

### DETAILED DESCRIPTION

Poor availability in the tissue of interest due to adverse physicochemical properties is a frequent cause of drug failure. Viruses overcome this constraint by embedding their nucleic acids into nanoscale particles and addressing them with high specificity to their target cells. While nanotechnology has provided a plethora of nanocarriers for drug transport, their ability to unequivocally identify cells of interest remained moderate. The present inventors herein show that particles endowed with a virus-like ability to identify cells by three consecutive checks for cell identity have a superior ability to identify mesangial cells in vivo compared to conventional nanoparticles. In mice this led to a 15-fold higher accumulation in the kidney mesangium followed by massive cell uptake. The present invention provides a surprisingly effective tool for transporting drugs into a target tissue, and this tool suitable for use in the treatment of various diseases, such as diabetic nephropathy for which currently no pharmacotherapy exists.

The present inventors designed particles (FIG. 1A) that carry, e.g. EXP3174 which is an angiotensin-II type 1 receptor (AT1R) ligand, in the NP corona to mediate receptor attachment. As a G protein-coupled receptor (GPCR) antagonist, it has the paramount advantage that binding cannot trigger cellular NP uptake, but only membrane binding and thus prevents particle uptake by off-target cells that only carry the AT1R.

For the second recognition criterion the present inventors outfitted the particles with the ability to probe cell surfaces for the presence of a target, such as angiotensin converting enzyme (ACE) which recognizes the proligand angiotensin-I (Ang-I) in the particle corona and converts it to the active ligand angiotensin-II (Ang-II).

As a third recognition step, a ligand such as Ang-II binds to a target, such as the AT1R, and, as an agonist, triggers cell uptake of particles upon receptor binding. The whole process of target cell recognition can best be illustrated with a flow chart (FIG. 1B). The particles were examined for their target receptor avidity and target-cell specificity in vitro. Additionally, it was assessed how the simultaneous presentation of two ligands addressing the same receptor, an antagonist promoting cell membrane binding, and an agonist, supporting cellular internalization, affects the NPs ability to mediate cellular uptake. Lastly, the present inventors showed that particles with such a virus-mimetic triple recognition strategy were superior to conventional NPs for reaching mesangial cells in vivo.

Since NPs are distributed in the organism typically by passive transport mechanisms, their appearance in a specific tissue is a matter of their physicochemical properties. However, the fraction of particles accumulating in a tissue of interest can be increased if they are able to actively interact with the cells of interest. It is not sufficient to outfit NPs with a ligand that binds to respective receptors to confirm a cell's identity. The particles of the present invention demonstrate clearly that strategies of a stepwise identification of cells, particularly strategies comprising a first ligand for mediating attachment to a target cell and a second ligand for mediating internalization of a nanoparticle into a target cell, are more advantageous.

Using viruses as a template, the present inventors designed NPs able to carry out a sequence of ‘if-then-else’ decisions that are taken one after another. In each single step, a NP probes a cell with the help of a ligand or substrate for the presence of a receptor or an ectoenzyme, respectively. If it is successful, the next identification step follows, if not (else) the particle ‘decides’ that the cell cannot be the target cell. Like viruses, this helps to avoid NP uptake by the ‘wrong’ cell type.

As an example, receptors that belong to the family of GPCRs, such as the AT1R, were used for decision making particles. In case cell identity is detected with a ligand such as a GPCR-antagonist, e.g. with EXP3174 or telmisartan, the positive outcome of this interaction (then) is that the particle binds to the cell surface and stays there. In case the next interaction fails to be positive (else), it is obvious that the particle is at risk of stranding on an off-target cell. However, in this case due to the thermodynamic equilibrium between free and bound particles, dropping concentrations of free particles as can be expected in vivo over time will shift the equilibrium such that the particles will dissociate with time from the ‘wrong’ target. In contrast, if the cell identity is detected with an agonist for the same GPCR, such as with Ang-II, the positive answer of the cell (then) is particle internalization. Thus, by carefully choosing the targets for the interaction and the type of ligand, the particles can be outfitted with a logic that may allow for an identification of even more concealed target cells than the exemplary targets investigated in this study. An example is a local ocular application in retinal tissue in which a particle is able to distinguish between the more than 60 cell types that are present, e.g. by targeting specifically endothelial cells.

The term “nanoparticle” and “NP”, as used herein, relates to a nanomaterial structure which comprises a first ligand and a second ligand. Particularly, a nanoparticle is a nano-object with all three external dimensions in the nanoscale, such as a liposome, a polymer nanoparticle, a micelle, a lipid nanocapsule, a liposome, an inorganic nanoparticle such as a gold nanoparticle or a Qdot. In one embodiment, the nanoparticles provide excellent biocompatibility and a highly tunable composition. Nanoparticles may be produced from a wide variety of materials, such as polymers, biomolecules, and metals. In one embodiment, a nanoparticle of the present invention is a virus mimetic nanoparticle that is capable of transporting a drug into a target tissue, such as in the mesangium of the kidney. In one embodiment, a nanoparticle comprises pirfenidon and/or cinaciguat and is for use in the treatment of diabetic nephropathy. In one embodiment, a nanoparticle of the present invention comprises biodegradable block-copolymers comprising PEG and PLA. In one embodiment, a first and a second ligand are covalently coupled to a nanoparticle via DCC/NHS or EDC/NHS. In one embodiment, said block-copolymers are solved in acetonitrile and mixed with PLGA (70/30, m/m) to obtain a polymer mixture. In one embodiment, nanoparticles are prepared using nanoprecipitation by injecting a polymer mixture dropwise into an aqueous phase. In one embodiment, the nanoparticles of the present invention accumulate in a target tissue, such as kidney tissue, within a very short time, i.e. <1 h, after administration of said nanoparticle.

In one embodiment, the nanoparticles of the present invention are very small, i.e. <80 nm, and, due to their small size, are able to rapidly exit the blood stream via fenestrated endothelium and to accumulate in a target tissue, such as mesangial tissue. In one embodiment, said nanoparticle further comprises a therapeutic agent, preferably any of pirfenidone and cinaciguat. In one embodiment, said particle has a size of from 5 nm to 1000 nm, preferably of from 10 nm to 150 nm, more preferably of from 20 nm to 100 nm. In one embodiment, the particle size is measured using dynamic light scattering, particle scattering diffusometry, nanoparticle tracking analysis, atomic force microscopy, or transmission electron microscopy, preferably dynamic light scattering or transmission electron microscopy. The size of a particle is determined by its diameter which relates to any straight line segment that passes through the center of a spherical particle and whose endpoints lie on the spherical particle. In cases of non-spherical particles, a diameter relates to the longest line segment that passes through the center of said non-spherical particle and whose endpoints lie on the particle. In one embodiment, a mean diameter relates to the mean of the diameters of nanoparticles comprised in a batch of nanoparticles. In one embodiment, the particles of the present invention have a polydispersity index of from 0.01 to 0.5, preferably from 0.01 to 0.3, more preferably from 0.01 to 0.1. In one embodiment, the polydispersity index is measured using dynamic light scattering, particle scattering diffusometry, nanoparticle tracking analysis, atomic force microscopy, or transmission electron microscopy, preferably dynamic light scattering or transmission electron microscopy. In one embodiment, the terms “nanoparticle” and “particle” are used interchangeably. In one embodiment, a nanoparticle of the present invention is for use in medicine. In one embodiment, the nanoparticle has a c-potential that is positive, negative, or neutral, for example of from −20 mV to 0 mV, or of from −15 mV to −5 mV. In one embodiment, the nanoparticle comprises at least one core comprising a nanomaterial and optionally a polymer and/or linker on its surface, wherein said first ligand and second ligand are coupled to said polymer and/or linker. In one embodiment, a particle has a polymer core.

The term “virus-mimetic”, as used herein, relates to an approach in which cell targeting approach of a virus is mimicked. A virus-mimetic particle of the present invention is being internalized by a target cell by means of a recognition process having at least two sequential steps. In such a two-step process, the first step is that a first ligand on a particle, such as EXP3174 or telmisartan, binds to a target which is expressed on a target cell, such as an angiotensin receptor on a mesangial cell. Subsequently, the particle is internalized into the target cell which is mediated by the second ligand that binds to a target on a target cell, such as activated angiotensin II or a cyclic amino acid sequence (cyclo Arg-Gly-Asp-D-Phe-Lys; SEQ ID No. 1), thereby initiating endocytosis of the particle. In one embodiment, a sequential presentation of the first and the second ligand is achieved by i) steric control mediated by using longer linkers, such as longer PEG-PLA chains, for the first ligand compared to shorter linkers, such as shorter PEG-PLA chains, used for the second ligand and/or by ii) an activation step in which a second ligand has to be activated to become capable of mediating internalization, such as a conversion of angiotensin I to angiotensin II. In one embodiment, the sequential presentation of ligands allows for a 15 times higher accumulation of the nanoparticles in target cells, such as mesangial cells, compared to conventional particles without ligand modification. In one embodiment, said particle has a particle avidity for a targeted receptor of from 1 pM to 100 nM, preferably 50 pM to 1 nM. In one embodiment, the nanoparticle of the present invention is for use as a medicament or diagnostic agent. In one embodiment, the nanoparticle of the present invention is for use in a method of preventing or treating a disease selected from diabetic nephropathy, glomerulonephritis, glomerular VEGF A dysregulation, endothelial VEGF A dysregulation, diabetic retinopathy, rheumatoid arthritis, age-related macular degeneration, and cancer such as breast cancer. In one embodiment, the terms “nanoparticle”, virus-mimetic particle”, and “decision-making nanoparticle” are used interchangeably.

The term “nanomaterial”, as used herein, relates to a material with any external dimension in the nanoscale or having internal structure or surface structure in the nanoscale. In one embodiment, said nanomaterial preferably comprises any of polyethylene glycol (PEG), polylactic acid (PLA), poly(lactic-co-glycolic acid) (PLGA), an oxazoline-derived polymer, a poly(aminoacid), a polysaccharide, a phospholipid, a sphingolipid, cholesterol, a PEG-lipid such as DPSE-PEG and acid stearic PEG, a block-copolymer such as PEG-PLA or PEG-poly-caprolactone, an inorganic substance such as gold or a qdot material, and a combination thereof. In one embodiment, said first ligand and said second ligand are each coupled to said nanomaterial, preferably are each coupled to a block-copolymer chain of said nanomaterial. In one embodiment, said nanomaterial comprises more than one block-copolymer chain, and said first ligand is coupled to a first block-copolymer chain of said nanomaterial and said second ligand is coupled to a second block-copolymer chain of said nanomaterial, and said first block-copolymer chain is longer than said second block-copolymer chain, preferably at least 1.5× longer than said second block-copolymer chain, more preferably at least 3× longer than said second block-copolymer chain. In one embodiment, said first block-copolymer chain comprises PEG in a range of from 1 k to 20 k, preferably 1 k to 10 k, and/or comprises PLA in a range of from 5 k to 40 k, preferably 10 k to 20 k. In one embodiment, a PEG chain of said first block-copolymer chain is longer than a PEG chain of said second block-copolymer chain. In one embodiment, one ligand is coupled to one PEG molecule. The term “k”, as used herein in the context of a polymer chain such as a block-copolymer chain, PEG, and/or PLA, refers to kilodalton (kDa). For example, PEG in a range of from 1 k to 20 k relates to PEG in a range of from 1 kDa to 20 kDa, e.g. PEG_{5kDa}. In one embodiment, PEG₅ₖ-PLA₁₀ₖ and PEG₂ₖ-PLA₁₀ₖ relate to PEG_{5kDa}-PLA_{10kDa} and PEG_{2kDa}-PLA_{10kDa}, respectively. In one embodiment, said first block-copolymer chain is PEG₅ₖ-PLA₁₀ₖ and said second block-copolymer chain is PEG₂ₖ-PLA₁₀ₖ. In one embodiment, said nanomaterial comprises a total amount of PEG (PEGₜₒₜₐₗ) and said particle has a ligand density of ligand/PEGₜₒₜₐₗ of at least 5%, preferably of at least 15%, more preferably of at least 25%, wherein the term “ligand” comprises both the first ligand and the second ligand. In one embodiment, the ligand density of ligand/PEG is ≤50%.

The term “first ligand”, as used herein, relates to a ligand that is capable of mediating an attachment of a nanoparticle to a target cell. In one embodiment, the first ligand merely initiates binding of the particle to the target cell and does not initiate internalization of the particle into the target cell, and the subsequent binding of the second ligand to the target cell is needed to initiate internalization of said particle into said target cell. In one embodiment, the first ligand is covalently or non-covalently coupled to a nanoparticle. In one embodiment, the first ligand is any biomolecule that triggers binding of the nanoparticle to the target cell, such as an antibody or antigen-binding fragment thereof, a peptide, an aptamer, a DNA nanostructure, a receptor ligand, and a receptor. In one embodiment, said first ligand is a non-agonistic agent binding to a GPCR, such as angiotensin II receptor type 1 (AT1r), human neuropeptide Y1-receptor, and C-X-C chemokine receptor type 4, and/or an agent binding to glycoprotein and/or glycolipid on a target cell surface, such as a heparan sulfate, a sialoglycoprotein, a ganglioside, and a mannose receptor, preferably is EXP3174 0r telmisartan. In one embodiment, a non-agonistic agent binding to a GPCR triggers binding of a particle to a target cell, but does not trigger internalization, such as endocytosis, of the particle into the target cell. In one embodiment, the first ligand and/or second ligand bind(s) to a target that is not ubiquitously expressed, but is predominantly expressed on a target cell in a target tissue. In one embodiment, the first ligand and/or second ligand bind(s) to a target structure on a target cell with an affinity K_{D}<100 nM. In one embodiment, the first ligand and/or second ligand has/have a molecular weight of <1500 Da. In one embodiment, the terms “target” and “target structure” are used interchangeably. In one embodiment, the term “target structure” relates to a protein, peptide, nucleic acid, saccharide, glycolipid, and/or glycoprotein presented on the surface of a target cell. In one embodiment, the first ligand is any AT1R antagonist with a free carboxylic acid residue for functionalization. In one embodiment, the first ligand is a selective AT1 antagonist, for example EXP3174 (losartan carboxylic acid) or telmisartan, which are potent and selective AT1 antagonists, or optionally a biologically active derivative thereof. In one embodiment, a “biologically active derivative” has the same biological function as EXP3174 or telmisartan, respectively, such as the same binding function and/or therapeutic function.

The term “non-agonistic agent”, as used herein, relates to an agent binding to a target structure which does not have an agonistic effect on said target structure, preferably which does not have an effect on said target structure mediating an internalization into a target cell.

The term “second ligand”, as used herein, relates to a ligand that is capable of mediating an internalization of a nanoparticle into a target cell. In one embodiment, the binding of the second ligand to a target structure, such as receptor, on a target cell initiates an internalization of the nanoparticle into the target cell. In one embodiment, a target structure on a target cell is any structure, such as a surface molecule, receptor, and/or biomarker that is typically expressed on the surface of a target cell. In one embodiment, the target structure on a target cell is a molecule that is typically overexpressed in a cell in a pathological condition compared to a healthy cell. In one embodiment, the first ligand and the second ligand target the same target structure on the target cell or target a different target structure on the target cell. In one embodiment, the second ligand is covalently or non-covalently coupled to a nanoparticle. In one embodiment, the second ligand is any biomolecule that triggers internalization of the nanoparticle into the target cell, such as an antibody or antigen-binding fragment thereof, a peptide, an aptamer, a DNA nanostructure, a receptor ligand, and a receptor. In one embodiment, said second ligand is any of i) an agent binding to an integrin, such as αVβ3 integrin, preferably selected from RGD, a cyclic RGD-peptide having a sequence of SEQ ID NO. 1, and derivatives thereof, ii) an agonistic agent binding to a GPCR such as AT1r, preferably activated angiotensin-II, iii) an agent binding to an ectoenzyme, such as legumain, a membrane-type matrix metalloproteinase, and angiotensin converting enzyme (ACE), preferably angiotensin-I, and/or iv) an agent binding to a transferrin-receptor. In one embodiment, said second ligand is enzymatically activated prior to said internalization of said nanoparticle into said target cell, for example angiotensin-I is activated to angiotensin-II by ACE prior to a binding of said second ligand to AT1r and internalization of said particle into said target cell. In one embodiment, an enzymatic activation of a second ligand is carried out by an ectoenzyme on the surface of a target cell, wherein preferably said enzymatic activation comprises enzymatic cleavage of said second ligand thereby providing an activated second ligand. In an alternative embodiment, said second ligand is not enzymatically activated prior to mediating internalization. In one embodiment, a ratio of said first ligand to said second ligand is in the range of from 2:1 to 1:2, preferably is 1:1. In one embodiment, the second ligand binds to said target cell after said first ligand binds to said target cell. In one embodiment, the sequential binding of said first ligand and said second ligand to said target cell increases the specificity to said target cell.

In one embodiment, if a second ligand targets AT1r, the second ligand being angiotensin-I is preferred over the second ligand being angiotensin-II, since the intermediate step of an enzymatic activation of angiotensin-I to angiotensin-II allows for increased specificity of the nanoparticles. In one embodiment, if the first ligand is EXP3174 and the second ligand is angiotensin-II or angiotensin-I to be activated to angiotensin-II, both the first ligand and the second ligand bind to the same target structure, namely to AT1R, on the target cell. In this embodiment, the first ligand which is EXP3174 binds to AT1R as an antagonist, and the second ligand which is, optionally after enzymatic activation, angiotensin-II binds to AT1R as an agonist. After the binding of the first ligand, such as EXP3174, there are still available AT1R binding sites for the second ligand. In one embodiment, the binding site of a target bound by a first ligand and the binding site of a target bound by a second ligand are different binding sites or are the same binding site but bound sequentially.

In one embodiment, the second ligand is shielded from binding to said target cell i) by steric hindrance, e.g. a block-copolymer chain or linker of the second ligand being shorter than a block-copolymer chain or linker of the first ligand, and/or ii) by the necessity of enzymatic activation of the second ligand prior to the second ligand being capable of mediating internalization.

The term “capable of mediating an attachment”, as used herein, relates to the binding ability of a first ligand to a target cell. The first ligand is capable of mediating an attachment, i.e. the first ligand triggers binding of a nanoparticle to a target cell, preferably by targeting a target structure on the target cell. In one embodiment, said binding of the first ligand to the target cell does not trigger internalization of the nanoparticle into the target cell. In one embodiment, for an internalization of the nanoparticle into the target cell, a further interaction, namely an interaction of the second ligand with the target cell is needed, preferably comprising the second ligand targeting a target structure on the target cell, which is the same or different from the target structure of the first ligand. In one embodiment, the first and second ligand may bind to the same target structure, but to different sites of the target structure, e.g. an agonist binding site and an antagonist binding site.

The term “capable of mediating an internalization”, as used herein, relates to the ability of a second ligand to trigger internalization of a nanoparticle into a target cell. In one embodiment, a second ligand binds to a target structure on a target cell thereby initiating internalization of the nanoparticle into the target cell. In one embodiment, said ability to trigger internalization may comprise the ability to be activated prior to triggering internalization, e.g. by enzymatic activation. In one embodiment, said internalization involves any of receptor mediated endocytosis, clathrin-coated pits, and/or caveolae.

The term “target cell”, as used herein, relates to a cell that is involved in a pathological condition, i.e. a disease. In one embodiment, a treatment of a disease comprises targeting a cell that is involved in said disease with a nanoparticle of the present invention. In one embodiment, the nanoparticle of the present invention serves as a drug delivery system which transports a drug to said target cell. In one embodiment, said target cell is selected from a mesangial cell, an endothelial cell, such as a retinal endothelial cell, and a tumor cell.

The term “therapeutic agent”, as used herein, relates to any substance intended for medical treatment. In one embodiment, a therapeutic agent is comprised in a particle main body, i.e. core, of a nanoparticle, such as in the interior of said nanoparticle and/or throughout the particle nanomaterial, and/or is coupled to said nanoparticle using a linker. In one embodiment, said therapeutic agent is a lipophilic therapeutic agent and is encapsulated and/or comprised by the particle main body. In one embodiment, a therapeutic agent is non-covalently or covalently coupled to said particle, e.g. by a cleavable linker. In one embodiment, said therapeutic agent may be covalently or non-covalently coupled to any component of the nanoparticle. In one embodiment, said therapeutic agent is an antifibrotic agent or a chemotherapeutic agent. In one embodiment, said therapeutic agent is any of pirfenidone and cinaciguat. The term “particle main body”, as used herein, relates to the main supporting structure of a particle. For example, a particle main body may relate to a lipid bilayer of a liposome or to a core structure of a polymer and/or solid-lipid particle. In one embodiment, a nanoparticle of the present invention is loaded with a therapeutic agent for specifically treating a target cell with the therapeutic agent, such a mesangial cell. In one embodiment, a therapeutic agent to be loaded into a particle, such as pirfenidon or cinaciguat, is dissolved in the polymeric phase, and is incorporated into said particle during preparation of a particle polymer core, and/or is dissolved in an aqueous phase comprised by a particle, such as an aqueous phase comprised by a liposome, and/or is dissolved in a lipid phase comprised by a particle, such as a lipid phase comprised by a liposome. Pirfenidon is a TGF-beta antagonist and has been proposed as a candidate for treating mesangial-associated pathological fibrosis. Cinaciguat (BAY 58-2667) is a soluble guanylate cyclase (sGC)-activator, and has been proposed for treating diabetic nephropathy. In one embodiment, the therapeutic agent pirfenidon or cinaciguat is efficiently incorporated into the polymeric core of a nanoparticle without significantly changing the properties of the particle, which is possible due to the lipophilic properties of the therapeutic agent. In one embodiment, said nanoparticle is for use in the treatment of diabetic nephropathy and said therapeutic agent is an antifibrotic agent. In one embodiment, said nanoparticle is for use in the treatment of cancer and said therapeutic agent is a chemotherapeutic agent.

The present invention further relates to a composition comprising a nanoparticle of the present invention and a pharmaceutically acceptable excipient. The nanoparticles of the invention can be admixed with suitable auxiliary substances and/or additives to obtain a pharmaceutically acceptable composition. Such substances comprise pharmacological acceptable substances, which increase the stability, solubility, biocompatibility, or biological half-life of the nanoparticles or comprise substances, which have to be included for certain routs of application like, for example, intravenous solution, sprays, band-aids or pills. The present invention also relates to a composition comprising a nanoparticle of the present invention, for use in medicine, e.g. for use in a method of preventing or treating a disease selected from diabetic nephropathy, glomerulonephritis, glomerular VEGF A dysregulation, endothelial VEGF A dysregulation, diabetic retinopathy, rheumatoid arthritis, age-related macular degeneration, and cancer such as breast cancer.

The nanoparticle of the present invention uses a novel virus mimetic recognition principle for a target cell, such as a mesangial cell, which results in highly efficient accumulation of the nanoparticle in a target tissue, such as mesangium. In one embodiment, the nanoparticle is combined with a suitable therapeutic agent, i.e. the therapeutic agent is incorporated into the nanoparticle and/or linked to the nanoparticle, which allows for targeted therapy of target cells in a target tissue, such as mesangial cells in mesangium. In one embodiment, a nanoparticle comprising a therapeutic agent is used for preventing or treating diabetic nephropathy. In one embodiment, the nanoparticle of the present invention allows for targeting a target cell using a recognition process having at least two steps. In one embodiment, the nanoparticle of the invention comprises a selective AT1 antagonist, e.g. EXP3174, as a first ligand and further comprises a therapeutic agent, preferably pirfenidone and/or cinaciguat. In one embodiment, the nanoparticle of the invention is a EXPcRGD nanoparticle, i.e. a nanoparticle that comprises EXP3174 as a first ligand and cRGD, particularly cRGDfK, as a second ligand, or the nanoparticle is a EXPAng-I nanoparticle, i.e. a nanoparticle that comprises EXP3174 as a first ligand and Ang-I as a second ligand. In one embodiment, said EXPcRGD nanoparticle and/or EXPAng-I nanoparticle further comprise(s) a therapeutic agent, preferably pirfenidone and/or cinaciguat. In one embodiment, said nanoparticle is a nanoparticle comprising a selective AT1 antagonist, e.g. EXP3174 as a first ligand, preferably a EXPcRGD nanoparticle, and further comprises a therapeutic agent which is cinaciguat. In one embodiment, said nanoparticle comprises a nanomaterial that comprises or consists of PLGA and/or PEG-PLA.

The term “administering”, as used herein, relates to the administration of an agent, e.g. a nanoparticle of the present invention and/or a pharmaceutical composition of the present invention, which can be accomplished by any method which allows the agent to reach the target cells. These methods include, for example, injection, oral ingestion, inhalation, nasal delivery, topical administration, deposition, implantation, suppositories, or any other method of administration where access to the target cells by the nanoparticle is obtained. An injection may relate to an intravenous, intradermal, subcutaneous, intramuscular or intraperitoneal injection. An implantation may include inserting implantable drug delivery systems comprising a nanoparticle of the present invention and/or may relate to hydrogels comprising nanoparticles, particularly hydrogels that are injected subcutaneously and/or intraperitoneally which gel in situ and which retardedly release nanoparticles. Suppositories include glycerin suppositories. Inhalation includes administering the nanoparticle with an aerosol in an inhalator, either alone or attached to a carrier that can be absorbed. The nanoparticle can be suspended in liquid such as in colloidal form.

An “effective amount” is an amount of the nanoparticle or the pharmaceutical composition that alleviates symptoms as found for the disease and/or condition.

The term “patient”, as used herein, relates to a human or an animal, preferably a mammal. Treating of a patient is meant to include, e.g., preventing, treating, reducing the symptoms of, or curing the disease or condition, for example cancer or diabetic nephropathy.

The term “block-copolymer”, as used herein, relates to a polymer comprising two or more homo- or copolymer subunits linked by covalent bonds. An intermediate non-repeating subunit may be comprised which is a junction block. Block copolymers are made up of blocks of different polymerized monomers. The term “block-copolymer chain” relates to a chain of a block-copolymer.

The term “DCC/NHS-coupling” and “EDC/NHS-coupling”, as used herein, relate to coupling reactions. A common way to synthesize an NHS-activated molecule is to mix NHS with, e.g. a desired carboxylic acid, and a small amount of an organic base in an anhydrous solvent. A coupling reagent such as dicyclohexylcarbodiimide (DCC) or ethyl(dimethylaminopropyl) carbodiimide (EDC) is then added to form a highly reactive activated intermediate. In one embodiment, the first ligand and the second ligand are coupled to said nanomaterial in at least two steps. In one embodiment, the first ligand and the second ligand are coupled to said nanomaterial via a linker, via a fusion protein, and/or via PEG.

The present inventors herein successfully show that virus-mimetic NPs that double/triple check cell identity allow for an enhanced NP accumulation in the targeted MCs in vivo. By combining an antagonistic ligand, mimicking initial cell attachment of viruses, with an enzyme mediated target cell recognition process, the particles had an outstandingly high in vitro target avidity together with an exceptional target cell specificity. The present inventors also demonstrate that the simultaneous hetero-multivalent binding of a particle-tethered agonist and antagonist for the same GPCR surprisingly leads to particle uptake. Overall, non-specific size-mediated passive targeting is not sufficient to achieve a satisfactory particle accumulation in MCs. Even traditional particle functionalization with a single ligand appears to be an insufficient approach. However, by mimicking the intricate multistep viral target cell binding and recognition process the present inventors obtained particles that are able to identify and accumulate in MCs. This opens new options for the delivery of drugs for the treatment of various diseases including renal diseases.

The present inventors further manufactured adenovirus-mimetic block-copolymer nanoparticles that effectively targeted glomerular mesangial cells due to a sterically controlled, sequential ligand-receptor interaction. Hetero-multivalent NPs thereby not only showed precisely tunable physicochemical characteristics, but also displayed excellent avidity for both target motifs, leading to a substantial AT1r binding in the picomolar range and a significantly increased mesangial cell uptake compared to unfunctionalized NPs. Profiting from these features, virus-mimetic NPs could selectively target mesangial cells, even in a surrounding environment of off-target cells. Additionally, hetero-multivalent NPs displayed the necessary in vivo robustness, leading to an efficient accumulation in mesangial areas in vivo with only marginal off-target deposition within the kidney. Remarkably, hetero-multivalent EXPcRGD NPs thereby showed far better mesangial targeting compared to homo-functional cRGD or EXP NPs. As the present inventors were able to target the same distinct cell type in vivo with two divergent virus-inspired concepts, the present inventors conclude that mimicry of viral infection patterns allows for highly effective targeting concepts. Moreover, the successful mesangial cell targeting allows for a refined therapy of mesangium-related kidney pathologies since it dramatically increases drug delivery compared to all other currently available approaches.

### BRIEF DESCRIPTION OF THE FIGURES

The present invention is now further described by reference to the following figures.

All methods mentioned in the figure descriptions below were carried out as described in detail in the examples.

FIG. 1 shows virus-mimetic attachment and target cell recognition.

(A) NPs carrying EXP3174 and Ang-I on their corona (NPEXPAng-I) attach to the cell membrane through EXP3174-mediated AT1R-binding. Specific recognition is triggered through enzymatic Ang-I processing and Ang-Il-mediated internalization.

(B) Flow chart exemplifying the triple target cell recognition of decision-making NPs.

FIG. 2 shows nanoparticle characterization.

(A) Assembly of ligand-decorated NPs.

(B) Molar ligand content of different NP species normalized to the PEG content,

(C) size and polydispersity index (PDI) and

(D) c-potential of the resulting NP formulations.

Results are presented as mean±SD of at least n=3 measurements.

FIG. 3 shows in vitro interaction with the AT1R and ACE.

Interaction of ligand-decorated NPs with their target ATTR (A-D) and ACE (E-F) determined by intracellular calcium measurements. (A) Ligand affinity and (B) particle avidity for the AT1R. (C) IC50 values for the free and particle-bound ligands. (D) Kinetic measurement of the AT1R inhibition by ligand-decorated particles. (E) Michaelis-Menten kinetics of NPEXPAng-I and NAng-I. (F) Specificity constant (Kcat/km) for the free and particle-bound Ang-I calculated based on ligand and NP concentration. Results are presented as mean±SD of at least n=3 measurements. Levels of statistical significance are indicated as **p≤0.01, ***p≤0.001, ****p≤0.0001 and #p≤0.0001 and x≤0.001 comparing the AT1R inhibition of NPEXP and NPEXPAng-I at different time points. n.s.: non-significant.

FIG. 4 shows Cellular internalization of NPEXPAng-I (red) in target rat mesangial cells (rMCs) cells (white) transfected with a YFP-tagged AT1R (green) (pAT1R-rMCs) at different incubation times. Scale bar 20 μm.

FIG. 5 shows uptake specificity of virus-mimetic NPEXPAng-I.

(A) Ligand-mediated internalization of NPEXPAng-I, NPAng-I and NPEXP in rMCs inhibited by free EXP3174 and captopril (see also FIGS. 11 and 12) (B) uptake of NPEXPAng-I in AT1R and ACE positive rMCs and HK-2 cells and AT1R and ACE negative HeLa cells. Specificity of particle uptake in co-culture of target rMCs with off-target (C) NCI-H295-R cells or (D) HeLa cells analyzed via flow cytometry. (E) CLMS images of particle uptake (red) in green-stained (CTG) rMCs (green) in co-culture with deep red-stained (CTDR) off-target HeLa or NCI-H295R cells (white). Scale bar 20 μm. (see also FIG. 13). Results are presented as mean±SD of at least n=3 measurements. Levels of statistical significance are indicated as **p≤0.01, ***p≤0.001, ****p≤0.0001 and as #p≤0.0001 comparing the uptake of NPs in cells with and without captopril or EXP3174 inhibition. n.s.: non-significant.

FIG. 6 shows NP distribution in mice kidney. (A) NPEXPAng-I fluorescence located in the kidney glomeruli (white arrows) (B) Control, non-targeted NPMeO do not accumulate in the kidney glomeruli, which lack particle-associated fluorescence. Blue: DAPI staining of cell nuclei; Green: Tissue autofluorescence; Red: NP-associated fluorescence. From left to right squared out regions are shown as magnifications.

FIG. 7 shows an assessment of the NP-associated fluorescence detected in kidney glomeruli analyzed through fluorescence microscopy.

(A) Images of kidney glomeruli (dotted circles) of mice treated with the different particle formulations. Scale bar 40 μm. see also FIG. 14B (B) Quantitative analysis of the complete glomerular NP-fluorescence. (C) Comparison of the particle-associated fluorescence in the glomeruli of the outer and inner cortex. (D) Glomerular localization of NPEXPAng-I determined via CLSM and Integrin-α8-staining of MCs (Scale bar 20 μm). Results in (C) and (D) are presented as mean±SD of at least n=120 fluorescence measurements of n=6 mice per NP sample. Levels of statistical significance are indicated as ****p≤0.0001. n.s.: non-significant.

FIG. 8 shows ligand coupling to PEG-PLA block copolymers.

(A) Lys-Ang-I and (B) EXP3174 were linked to carboxylic acid- or amine-ended PEG₅ₖ-PLA₁₀ₖ using EDC/NHS or DCC/NHS chemistry, respectively. (C) Complete polymer functionalization shown by the quantification of the molar ligand and PEG content. (D) Absence of unreacted NH₂ polymer end groups on EXP3174-modified-polymer was determined using flurescamine. A Student's t-test was performed using GraphPad Prism 6.0 to assess statistical significance. Levels of statistical significance ae indicated as ****p≤0.0001 comparing the fluorescence of MeO—and EXP3174—with NH₂-terminated PEG₅ₖ-PLA₁₀ₖ. For detailed methods see Example 1.

FIG. 9 shows maximum calcium signal and inhibition by NPEXP.

rMCs were stimulated simultaneously with Ang-II and NPEXP and the resulting intracellular calcium response measured immediately for 1 minute. At the used NPEXP concentrations, the EXP3174 ligand did not inhibit the agonist-triggered calcium signal during the assay duration. Therefore, the influence of EXP3174 on the Ang-II-measurement was considered negligible. Results are shown as mean±SD of at least n=3 measurements. For detailed methods see Example 1.

FIG. 10 shows the uptake of different particle formulations over time in AT1R positive pAT1R-rMCs analysed through CLSM.

(A) NPEXP are not internalized in the cell line and mostly locate on the cellular membrane and filipodia between cells forming big clusters over time. Receptor binding is shown by the colocalization of NP- and receptor-associated fluorescence. (B) NPAng-I are internalized by the cells as depicted by their cytoplasmic localization. (C) NPMeO do not associate with cells due to their lack of a tethered ligands enabling a specific targeting. Cells: white; AT1R-YFP: green; NP-formulations: red. Scale bar 20 μm. For detailed methods see Example 1.

FIG. 11 shows that EXP3174 counterbalances the uptake decrease due to steric hindrance of the Ang-I ligand by long polymer chains on NPEXPAng-I.

NPAng-I (grey) with 20% Ang-I density were prepared with varying polymer densities of COOH-PEG₅ₖ-PLA10k and analysed for their cellular uptake using flow cytometry. Concomitantly, NPEXPAng-I (yellow) were prepared with varying densities of EXP3174-PEG₅ₖ-PLA10k to compare the effect of the second ligand on the stearic hindrance of Ang-I. Functionalization of long polymer chains with EXP3174 on NPEXPAng-I counterbalanced the decreased uptake due to stearic hindrance of the Ang-I ligand when adding non-functionalized long polymers, and significantly increased the particle internalization. Results are presented as mean±SD of at least n=3 measurements. A 2-way ANOVA with Sidak's multiple comparisons test was performed using GraphPad Prism 6.0 to assess statistical significance. Levels of statistical significance ae indicated as ****p≤0.0001. For detailed methods see Example 1.

FIG. 12 shows the specificity of the NP uptake analysed through CLSM.

Cells were preincubated for 30 minutes with free EXP3174 prior to the addition of the different NP formulations (NPEXPAng-I, NPAng-I and NPEXP). Inhibition of the target receptor resulted in the suppression of the particle-associated fluorescence. Scale bar 20 μm. For detailed methods see Example 1.

FIG. 13 shows the uptake of (A) NPEXP and (B) NPAng-I in co-culture of target and off-target cells.

NPEXP show accumulation in rMCs and NCI-H205R cells, as they both carry the AT1R. Contrary, the co-culture of rMCs and HeLa cells shows preferential accumulation of NPEXP in rMCs, as HeLa cells express only minor amounts of the receptor on the cell membrane (cf. [3]). NPAng-I show a higher specificity as they preferentially accumulate in target rMCs, which carry the necessary equipment for their internalization (the ACE and the ATTR), over off-target cells lacking ACE (HeLa or NCI-H295R cells). Cell nuclei: blue; Off-target cells (HeLa or NCI-H295R): white; Target cells (rMCs): green; NPs: red. Scale bar 20 μM. For detailed method see Example 1.

FIG. 14 shows in vivo distribution of different NP formulations.

(A) Kidney distribution of NPAng-I and NPEXP in mice kidneys. NPAng-I show a small NP-associated fluorescence in the majority of glomeruli contrary to NPEXP which did not accumulate in this area. The glomeruli are marked with white arrows for better visualization. From left to right squared out regions are shown as magnifications. DAPI staining of cell nuclei: blue; Tissue autofluorescence: green; NP-associated fluorescence: red. (B) Kidney distribution of the free dye used to label the NPs. CF647 was injected into mice as a control to assess its distribution in the kidney. Strong fluorescence could be detected in the tubular area, with no fluorescence in the glomeruli (marked with a white circle). This demonstrates that the fluorescence seen for the particle-samples comes from the particles themselves and not from leaked dye, which is freely filtrated due to its low molecular weight. (C) Plasma residence of different NP-formulations after one-hour circulation in NRMI mice. NP fluorescence in plasma 1 hour after injection was measured and normalized to the fluorescence measured 5 minutes after injection (initial particle blood fluorescence). Non-targeted NPs (NPMeO) show the highest blood circulation time, which is due to the stealth effect conferred by their PEG-shell. Even though 40% of all the polymers on the NPEXPAng-I surface are ligand-coupled, which decrease the particle stealth effect, they are able to match the blood residence of non-targeted particles. They depict a significant higher fluorescence in plasma after 1 h compared to particles functionalized with only one ligand (NPAng-I and NPEXP). NPAng-I, which carry a specific two-step virus-mimetic recognition mechanism also show a significant superior blood residence than NPEXP, which represent commonly targeted NPs. As a control, the free dye used to label the particles (CF647) was additionally injected into mice and it rapidly disappears from the blood circulation after its to its free filtration (6% of the initial fluorescence after 1 h). Results in (C) are presented as mean±SD of at least n=6 samples. A Student t test was performed using GraphPad Prism 6.0 to assess statistical significance. Levels of statistical significance are indicated as *p≤0.05, ***p≤0.001 and ****p≤0.0001. n.s.: non-significant. For detailed methods see Example 1.

FIG. 15 shows an exemplary in vivo use of decision-making nanoparticles of the present invention which comprise a first ligand for attachment to a target cell and a second ligand for recognition/internalization of the nanoparticle into said target cell. A nanoparticle of the present invention can be used for targeting various target tissues, for example mesangium. The nanoparticles can be customized for targeting specific target tissues by choosing a first and a second ligand targeting a target receptor and/or molecule on the surface of a target cell.

FIG. 16 shows transversal kidney sections of animals treated with particles.

A) virus mimetic particles show high accumulation in the renal corpuscles (round labelling).

B) unmodified nanoparticles, which do not carry the first ligand and the second ligand, do not show significant uptake into glomerular tissue.

FIG. 17 shows that adenovirus-mimetic NPs enter glomerular mesangial cells via a synergistic combination of passive and active targeting strategies. (a) Upon administration, NPs reach glomerular areas of the renal filtration system via the afferent arteriole that then diverges into the glomerular capillary system. (b) Within the glomerular capillaries, NPs cannot pass the renal filter due to its meshwork-like structure, but are able to extravasate through endothelial fenestrations, thereby reaching the interstitially located mesangium. (c) Thereupon, virus-mimetic NPs can effectively infiltrate mesangial cells via initial binding to Angiotensin II receptor type 1 (AT1r) and subsequent αVβ3 integrin-mediated endocytosis. (AA: afferent arteriole; EA: efferent arteriole; MC: mesangial cells; PO: podocyte; FP: foot processes; ET: endothelium; BM: basement membrane.)

FIG. 18 shows a characterization of adenovirus-mimetic NPs. (a) Particle design of hetero-multivalent EXPcRGD NPs as well as homo-functional or unfunctionalized NP species. (b) DLS analysis. All particle types were manufactured below a size threshold of 60 nm without considerable aggregation. (PDI: polydispersity index.) (c) Zeta potential measurements. Addition of COOH-PEG₂ₖ-PLA₁₀ₖ to the polymer mix results in negative surface charges. (d)/(e) Quantification of cRGDfK and EXP3174 ligand surface density, respectively, after NP manufacture. Final surface content was directly proportional to the priorly added amount of ligand-functionalized polymer for both cRGDfK (R²=0.9957) and EXP3174 (R²=0.9871). Results represent mean±SD (n=3).

FIG. 19 shows AT1r interaction of adenovirus-mimetic NPs. (a) Intracellular calcium levels after AT1r stimulation of rMCs treated with free or particle-bound EXP3174. Both EXPcRGD NPs (IC₅₀=276±31 pM) and EXP NPs (IC₅₀=552±73 pM) effectively bound and thereby inhibited the AT1r, resulting in low Ca2+ influx upon AT1r stimulation with Angiotensin II (ATII). This effect was even stronger than for free EXP3174 (IC₅₀=2.66±0.9 nM), due to a multivalency-derived avidity gain. (b) AT1r activity for rMCs treated with AT1r ligand-free NPs. Neither unfunctionalized Control NPs nor cRGD NPs could significantly bind the AT1r, resulting in maximum calcium levels upon ATII stimulation and confirming the specificity of the assay. (M=molar concentration of either NPs or EXP3174.) Results represent mean±SD (n=3).

FIG. 20 shows an internalization of NPs by target mesangial cells in vitro. (a) CLSM analysis of CTDR-stained rMCs after incubation with 0.05 mg mL⁻¹ of AlexaFluor™568-labeled EXPcRGD nanoparticles. NP-associated fluorescence (purple) could be detected in vesicular structures within the rMC cytosol (grey). With increasing incubation time, endocytotic vesicles grew both in size, number and intensity, indicating fusing of vesicles into larger endosomes. (Scale bar 20 μm.) (b) Flow cytometry analysis of NP uptake into rMCs. Hetero-multivalent EXPcRGD NPs showed a substantially increased cell-uptake compared to Control NPs as well as homo-functional NPs. (c) Flow cytometry results after 60 min NP incubation. While EXPcRGD NPs showed maximum cell-association compared to all other NP types, addition of an excess of free cRGDfK (c=500 μM) sharply decreased fluorescence signals to the level of EXP NPs, indicating αVβ3-dependency of NP uptake. Results represent mean±SD (n=3). ⬆P<0.0001, 0P<0.001, **** P<0.0001. (AFU, arbitrary fluorescence units.)

FIG. 21 shows TEM analysis of NP interaction with mesangial cells. (a) EXPcRGD NPs accumulated in numerous vesicular structures (black arrows) within rMCs. Vesicles of differing sizes were present both in outer and inner parts of the cell cytosol, indicating intracellular processing and fusing into larger endosomes. Moreover, a substantial number of NPs was still located at the cell membrane, suggesting that particle underwent a stepwise process of initial cell binding and subsequent endocytosis. (Images on the right show zoomed-in view of black boxes on the left.) (b) EXP NPs, in contrast, merely accumulated at the cell border where they bound to distinct surface structures of rMC cells, indicating a possible interaction with membrane-bound AT1r. (Image in top left corner shows zoomed-in view of black box.) (c) Control NPs showed only negligible interaction with rMCs with hardly any gold-enhanced NPs visible.

FIG. 22 shows mesangial cell selectivity of EXPcRGD NPs in an in vitro co-culture assay. (a) CLSM analysis of CTG-stained rMCs (green), co-cultured with CTDR-labeled HeLa or NCI-H295R cells (grey), which acted as off-target cells. Cell nuclei were stained with Hoechst 33258 (blue). For the rMC/HeLa co-culture (top row), NP-derived fluorescence (purple) could merely be detected within areas of rMCs, indicating a selective EXPcRGD NP uptake into the target cells. Co-culture of rMCs with AT1r-expressing NCI-H295R cells lead to a diverging NP distribution (bottom row). EXPcRGD NPs thereby also bound to the surface of NCI-H295R cells, leading to a diffuse pattern around the cell border. However, efficient particle uptake into circular endocytotic vesicles could only be seen in mesangial cells. (Scale bars 20 μm.) Flow cytometry analysis of rMCs with both HeLa (b) and NCI-H295R (c) supported CLSM results as EXPcRGD NPs showed a significantly higher cell-association with rMCs in both cases. Interestingly, addition of an excess of free EXP3174 (c=1 mM) in (c) sharply reduced NP interaction with NCI-H295R cells, indicating that EXPcRGD NPs could no longer bind to NCI-H295R cells via AT1r. Results represent mean±SD (n=3). **** P<0.0001. (n.s.: not significant. AFU, arbitrary fluorescence units.)

FIG. 23 shows that EXPcRGD NPs show strong intraglomerular accumulation in vivo. Transversal kidney cryosections were imaged using fluorescence microscopy. To facilitate histological evaluation, cell nuclei were stained with DAPI (blue) and tissue autofluorescence was recorded (green). EXPcRGD NPs (red) were found to have accumulated almost exclusively in glomerular areas of the cortex (white circles), while fluorescence in tubular areas was neglectable. (From top-left to bottom-right, images show zoomed-in views of white boxes.)

FIG. 24 shows that EXPcRGD NPs show a significantly enhanced accumulation in mesangial cells. (a) Fluorescence microscopy analysis revealed that high fluorescence levels within glomeruli (white circles) could mainly be detected for EXPcRGD NPs. While EXP NPs showed a moderate accumulation in glomeruli, Control NPs and cRGD NPs did not produce signals to any considerable extent. (Scale bar 20 μm. Calibration bar: 0-65535 Gray Value.) (b) Precise quantification of intraglomerular fluorescence intensity was achieved by assessing the integrated density per area of glomerulus for a sufficient number of glomeruli. Thereby, EXPcRGD NPs showed far higher fluorescence intensities per glomerulus compared to all other particle types. Results represent mean±SD (n=60). ****P<0.0001. (AFU, arbitrary fluorescence units.) (b) Antibody staining for mesangial surface marker integrin α-8 showed that EXPcRGD NP-associated fluorescence (red) was co-localized with areas covered by mesangial cells (yellow), indicating that EXPcRGD NPs were able to selectively infiltrate mesangial cells via endocytosis. Scale bar 20 μm.

FIG. 25 shows a synthesis concept for ligand-functionalized PEG-PLA block co-polymers. (a) Hetero-bifunctional PEG polymers (\textcircled{1}) of varying chain length (2 kDa/5 kDa) were mixed with 3,6-dimethyl-1,4-dioxane-2,5-dione (\textcircled{2}) to create NH₂-PEG₅ₖ-PLA10k as well as COOH-PEG₂ₖ-PLA₁₀ₖ (\textcircled{3}) via ring-opening polymerization. (b) Subsequently, NH₂-PEG₅ₖ-PLA₁₀ₖ was covalently coupled to the carboxyl group of EXP3174 (\textcircled{4}) via DCC/NHS chemistry, resulting in EXP3174-PEG₅ₖ-PLA₁₀ₖ (\textcircled{5}). (c) Additionally, COOH-PEG₂ₖ-PLA₁₀ₖ was attached to the lysine residue of cRGDfK (\textcircled{6}) via EDC/NHS chemistry, leading to shorter cRGDfK-PEG₂ₖ-PLA₁₀ₖ (\textcircled{7}). (d) Coupling efficiency for synthesized EXP3174-PEG₅ₖ-PLA₁₀ₖ was determined by comparing the molarity of both PEG and EXP3174. Molar concentration thereby did not significantly vary, indicating successful functionalization. (e) Coupling efficiency for cRGDfK-PEG₂ₖ-PLA₁₀ₖ was also in a reasonable range with merely negligible differences in molarity of PEG and cRGDfK. Results represent mean±SD (n=₃).

FIG. 26 shows gold-tagged NPs allowing for facilitated TEM visualization. NPs were gold-labeled by covalently attaching ultra-small gold nanoparticles (diameter: 2.2 nm) to the carboxyl group of PLGA, that was then used to manufacture NPs. After incubation of rMCs with labeled NPs, the particle core was gold-enhanced by depositing further gold particles on the NP core, thereby increasing electron density of the sample and enabling visualization in TEM microscopy, where NPs appeared as dark black spots.

FIG. 27 shows a quantification of αVβ3 expression by different cell types investigated by (a) Flow cytometry results and (b) CLSM. For cytometry analysis, unspecific binding sites were blocked with 2% BSA in DPBS and 105 cells were incubated for 1 h with a 1:20 dilution of AlexaFluor® anti-CD51/61 antibody in 0.1% BSA in DPBS (AlexaFluor® Mouse IgG1, κ Isotype Ctrl (FC) served as unspecific control). Thereafter, cells underwent several steps of DPBS washing and centrifugation. Finally, samples were resuspended in DPBS and analyzed using flow cytometry as previously described (FACS Calibur, excitation: 633 nm, emission: 661/16 nm bandpass filter). αVβ3-derived fluorescence levels were thereby maximal for rMCs while both HeLa and NCI-H295R cells showed negligible signals, that were both significantly lower than for rMCs. Results represent mean±SD (n=₃).****P<0.0001, **P<0.01, *P<0.05. (AFU, arbitrary fluorescence units.) To confirm flow cytometry results, rMCs were seeded into 8-well Ibidi slides (15.000 cells well-i) and stained for αVβ3 integrin as described above. Cells were then washed with DPBS, fixed with 4% PFA in DPBS and analyzed at a Zeiss LSM 710. CLSM images showed a strong integrin signal that was co-localized with rMC cell body, indicating a substantial αVβ3 expression by mesangial cells. Scale bars 20 μm.

FIG. 28 shows results of fluorescence imaging.

A) Relative blood plasma fluorescence after NP injection. Control NPs exhibited maximal blood circulation values with almost 50% residual blood plasma fluorescence after 60 min of injection. In contrast to EXP NPs as well as EXPcRGD NPs, that both showed tolerable residual concentrations, cRGD NPs were rapidly cleared from the blood. Results represent mean±SD (n=₃). ****P<0.0001, ***P<0.001, **P<0.01. (n.s.: not significant.)

B) Fluorescence imaging of kidney cryosections after injection of free CF™ 6₄₇ fluorescent dye. To visualize cell nuclei, sections were DAPI-stained (blue). After injection of a comparable molarity of free dye, strong fluorescence signals (red to white) could be detected in tubular areas of the kidney, indicating free renal filtration of the low-molecular dye. As expected, no intraglomerular accumulation could be detected (white circles). (Calibration bar: 0-65535 Gray Value.)

FIG. 29 shows a concept of NP-assisted cinaciguat delivery. Hetero-multivalent EXPcRGD NPs enter target renal mesangial cells via previously described sequential recognition sequence. After a successful endocytosis, NPs undergo endolysosomal degradation, leading to the release of cinaciguat (small dots). CCG then activates and stabilizes mesangial sGC, leading to an enhanced, NO-mediated production of cGMP. Via cGMP-mediated activation of protein-regulated kinase (PGK1-α), several pro-fibrotic pathways are inhibited, leading to an overall reduction in pro-fibrotic remodeling of mesangial cells.

FIG. 30 shows an exemplary experimental set-up. For all experiments, free cinaciguat (c=2 μM) was compared to EXPcRGD NPs carrying either CCG at a concentration of 0.2 μM or no drug. The amount of free cinaciguat was chosen in accordance with previous studies that had shown a potent effect in this concentration range.

FIG. 31 shows Western Blot analyses of cinaciguat effects on drug targets. a) sGC stimulation and stabilization. Both free CCG and CCG-loaded EXPcRGD NPs lead to a substantial increase in sGC levels during 24 h of incubation, while CCG-lacking control NPs showed no considerable effect. b) Activation of PGK1-α. To assess PGK1-αactivity, phosphorylation of its substrate vasodilator-stimulated phosphoprotein (VASP) was assessed. Both free CCG and CCG-carrying EXPcRGD NPs thereby showed a considerable increase in P-VASP/VASP ratio, while control NPs lead to no detectable changes. (n=3; ****P<0.0001; ***P<0.001; **P<0.01; *P<0.05).

FIG. 32 shows an antifibrotic and antiproliferative effect of cinaciguat-loaded NPs. Mesangial cells were pre-incubated with free CCG or NPs for 4 h prior to incubation with 10 ng mL⁻¹ of TGF-β for 48 h to induce fibrotic and hyperproliferative changes. a) MIT assay shows anti-proliferative effect. While mesangial cells showed a significant hyperproliferation upon TGF-β stimulation, pre-incubation with free CCG or CCG-loaded NPs considerably decreased this effect. Western Blot analysis of both b) a-SMA and c) Collagen I levels showed a drastic reduction of both fibrosis markers for CCG or CCG-loaded NPs compared to the TGF-β control. (n=3; ****P<0.0001; ***P<0.001; n.s. not significant). d) LSM analysis further supported the results from b) with mesangial cells showing a considerably reduced production of α-SMA upon pre-incubation with CCG or CCG-carrying NPs. Scale bar=2 μm.

In the following, reference is made to the examples, which are given to illustrate, not to limit the present invention.

### EXAMPLES

### Example 1: Materials and Methods

Cell culture

The cell lines used in this study were cultured at 37° C. and 5% CO₂. rMCs, NCI-H295R and HeLa cells were cultured in RPMI1640 medium (Sigma Aldrich) supplemented with 10% FBS and insulin-transferrin-selenium and 100 nM hydrocortisone. HK-2 cells were maintained in DMEM-F12 (1:1) medium (Sigma Aldrich) supplemented with 10% FBS. pAT1R-rMCs were obtained by transfecting rMCs with a plasmid encoding the AT1R with a YFP-tag (CXN2-HA-AT1R-YFP) (cf. [4]) using the commercially available transfection reagent Lipofectamine 2000 following the manufacturer's instructions. pAT1R-rMCs were cultured in RPMI1640medium supplemented with 10% FBS and 600 μg/ml geneticin (G418). The cell lines were characterized for their target AT1R and ACE expression as shown previously [1, 3].

Mice

The experimental procedures on animals were carried out according to the national and institutional guidelines and were approved by the local authority (Regierung von Unterfranken, reference number: 55.2-2532-2-329). The mice indicated in the Key Resources Table have been used in this study at the age of 10 weeks. Only female mice were used in all experiments. They were kept under Specific pathogen Free (SPF) housing facilities, under standard conditions (50±5% relative humidity, temperature of 21±1° C., air exchange >8 AC/h and light period of 12h:12h (L:D)).

Polymer Preparation: Block Copolymer Synthesis

PEG-PLA block-copolymers (COOH-PEG₂ₖ-PLA₁₀ₖ, COOH-PEG₅ₖ-PLA₁₀ₖ, NH₂-PEG₅ₖ-PLA₁₀ₖ, and MeO-PEG₅ₖ-PLA₁₀ₖ) were synthesized through ring opening polymerization of cyclic 3,6-dimethyl-1,4-dioxane-2,5-dione (lactide). In short, the lactide was recrystallized prior to use from anhydrous ethyl acetate and dried under vacuum at 40° C. for 12 hours at room temperature (r.t.). COOH-PEG₅ₖ-OH, COOH-PEG₂ₖ-OH, Boc-NH-PEG₅ₖ-OH or MeO-PEG₅ₖ-OH were used as macroinitiators for the ring opening polymerization. They were solved (0.3 mmol) in anhydrous DCM and mixed with the purified lactide (18 mmol). 1,8-diazabicyclo[5.4.0]undec-7-ene (DBU) (0.9 mmol) was added as a catalyst. The polymerization was quenched after 1 hour with benzoic acid (4.6 mmol). The resulting polymers were precipitated in diethyl ether and dried under vacuum at 40° C. (for COOH-PEG₂ₖ-PLA₁₀ₖ, COOH-PEG₅ₖ-PLA₁₀ₖ, and MeO-PEG₅ₖ-PLA₁₀ₖ) or 35° C. (for Boc-NH-PEG₅ₖ-PLA₁₀ₖ) for 12 hours. For the cleavage of the protective Boc group, Boc-NH-PEG-PLA was dissolved in 50% (v/v) TFA/DCM and stirred at r.t. for 30 minutes. Afterwards, it was diluted with the triple volume of DCM and washed with saturated sodium bicarbonate solution three times. The organic phase was dried over anhydrous sodium sulfate, filtered, and concentrated under reduced pressure. The resulting NH₂-PEG₅ₖ-PLA₁₀ₖ was purified through precipitation in diethyl ether and subsequently dried under vacuum at 35° C. for 12 hours.

Polymer Preparation: Ligand Coupling

For the preparation of Ang-I-modified polymers (see also FIG. 8) 14 μmol of COOH-PEG₅ₖ-PLA₁₀ₖ were activated with 25 molar excess of EDC and NHS in DMF for 2 hours. Afterwards, 863 μmol of 2-mercapthoethanol was added to quench the reaction for 20 minutes, prior to the dropwise addition of 66 μmol of DIPEA and 17 μmol of Lys-Ang-I in DMF. After 48 h, the resulting polymer was diluted in ultrapure water (Millipore) to a DMF concentration below 10% and dialyzed using a 6-8 kDa molecular weight cut-off regenerated cellulose dialysis membrane over 24 hours (with medium change after 30 minutes, 2, and 6 hours) to remove non-reacted ligand and reagents. For the preparation of EXP3174-modified polymer (see also FIG. 8) 96.4 μmol of EXP3174 were activated with an equimolar amount of DCC and NHS in DMF for 2 hours. Afterwards resulting urea byproducts were removed by centrifugation (5 min, 12,000×g) and subsequent filtration with a 0.2 μm Rotilabo PTFE syringe filter. 27.6 μtmol NH₂-PEG₅ₖ-PLA₁₀ₖ in DMF and a 17.5 molar excess of DIPEA were added to the activated ligand and reacted over 20 hours. The ligand-modified polymer was purified by precipitation in ice cold 1:5 (v/v) diethyl-ether:methanol and subsequent dialysis against 10% ethanol in 10 mM borate buffer (pH 8.5) for 24 hours to remove excess free ligand (with medium change after 30 minutes and 6 hours) followed by dialysis against ultrapure water to remove buffer salts over 24 hours (with medium change after 30 minutes, 2 and 6 hours) using a 6-8 kDa molecular weight cut-off regenerated cellulose dialysis membrane (Spectrum Laboratories). Ligand-modified block-copolymers were lyophilized over 72 hours prior to ligand-coupling confirmation. For that, polymers were solubilized in ACN at a concentration of 40 mg/ml and precipitated in stirring ultrapure water to create polymer micelles (final concentration 1 mg/ml). The PEG content was quantified using a colorimetric iodine complexation assay and coupled Ang-I was quantified using a Pierce BCA assay kit following the manufacturer's instructions using a FLUOstar Omega microplate reader. EXP3174 was fluorescently quantified at λₑₓ=250 nm and λₑₘ=370 nm using a LS-5S fluorescence plate reader. The absence of unreacted NH₂ end groups on the NH₂-PEG₅ₖ-PLA₁₀ₖ was determined using flurescamine (See also FIG. 8).

Polymer Preparation: Fluorescence Labelling of PLGA

For in vitro and in vivo particle detection, fluorescently labelled PLGA was used in the particle core. To that end, TAMRA-amine (for CLSM) and CF6467-amine (for flow cytometry and in vivo experiments) were covalently coupled to carboxylic acid-terminated 13.4 kDa PLGA. Briefly, 5 μmol acid-terminated PLGA were dissolved in anhydrous DMF and activated for 2 h at r.t. with 129 μmol DMTMM (25-fold excess). Afterwards, 1 μmol fluorescent dye was dissolved in DMF, added dropwise to the PLGA and reacted for 72 h at r.t. in the dark. The reaction product was diluted (DMF<10%) and dialyzed against ultrapure water, using a 3.5 kDa molecular weight cut-off regenerated cellulose dialysis membrane (Spectrum Laboratories) over 34 hours (with medium change after 30 minutes, 2, and 6 hours) under light exclusion. Fluorescently labelled PLGA was then lyophilized over 3 days.

NP Preparation and Characterization: Particle Preparation

For NP preparation, PEG-PLA block-copolymers and 13.4 kDa PLGA were mixed at a 70:30 mass ratio to a final concentration of 10 mg/mL in ACN. For ligand-modified particles COOH-PEG₂ₖ-PLA₁₀ₖ and ligand-modified polymers were mixed accordingly so that 20% of the polymers making up the NP-structure were modified with Ang-I (NPAng-I) or/and EXP3174 (NPEXP and NPEXPAng-I, respectively). NPs were prepared via bulk nanoprecipitation of polymer mixtures in vigorously stirring 10% DPBS (v/v) (pH 7.4) to a final concentration of 1 mg/ml. Particles were stirred for 2 hours to ensure the evaporation of the organic solvent, and concentrated by ultracentrifugation using a 30-kDa molecular weight cutoff Microsep advance centrifugal device (Pall Life Sciences) for 20 minutes at 756 g.

NP Preparation and Characterization: Dynamic Light Scattering and ζ-Potential

Size and ζ-potential of the resulting particles were determined in 10% PBS at a constant temperature of 25° C. using 1 mg/mL or 3.5 mg/ml concentrations, respectively, with a ZetaSizer Nano ZS (Malvern Instruments) equipped with a 633 He—Ne laser at a 173° backscatter angle and the Malvern Zetasizer software version 7.11. The cuvette position was set at 4.65 mm and the attenuator optimized automatically by the device. Disposable microcuvettes (Brand) and a folded capillary cell (Malvern Instruments) were used for size and 4-potential measurements, respectively.

NP Preparation and Characterization: Particle Quantification

Quantification of particle PEG concentration was performed using a colorimetric iodine complexing assay and correlated with the gravimetrical NP content determined via lyophilization. In short, the particle samples were diluted in ultrapure water to a PEG concentration in the 5-30 μg/mL range. Dilutions in ultrapure water of MeO-PEG-OH (0-40 μg/mL) were used as standards for the calibration curve. 140 μL of samples or standards were mixed with 60 μL of a 2:1 (v/v) mixture of 5% (m/v) barium chloride solution in 1 N HCl and a 0.1 N aqueous iodine solution. The samples and standards were transferred into a 96-well plate and their absorbance at 535 nm measured using a FUOstar microplate reader (BMG Labtech). The correlation of the particle PEG content with the exact polymer concentration was determined gravimetrically after sample lyophilization. The molar particle concentration was calculated from the particle mass determined through the colorimetric iodine complexing assay, the particle density (1.25 g/cm³) and the hydrodynamic diameter of the NPs obtained through DLS measurements assuming a spherical particle shape. Ligand concentration on the particle corona was quantified using a BCA assay, and fluorometrically for Ang-I and EXP3174, respectively, as described above.

Intracellular Calcium Measurements

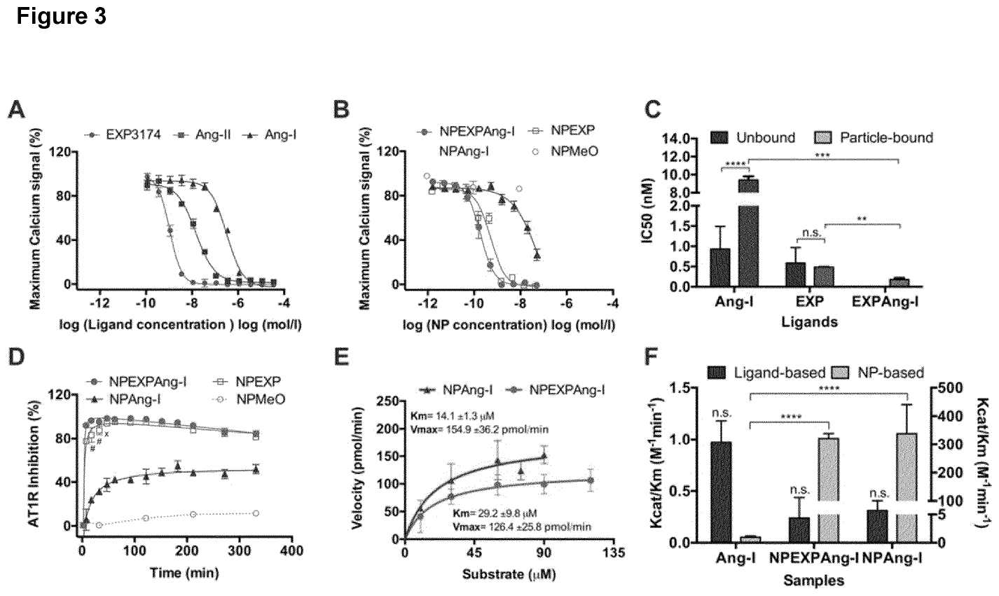

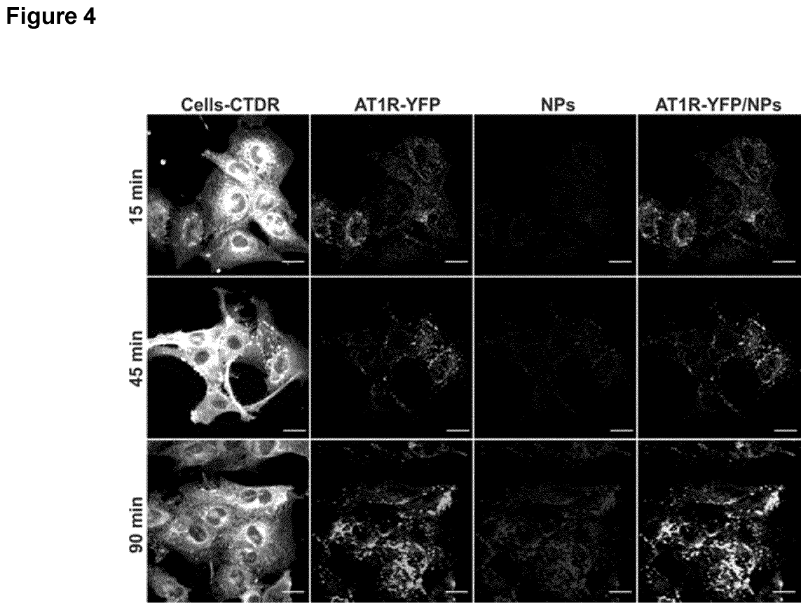

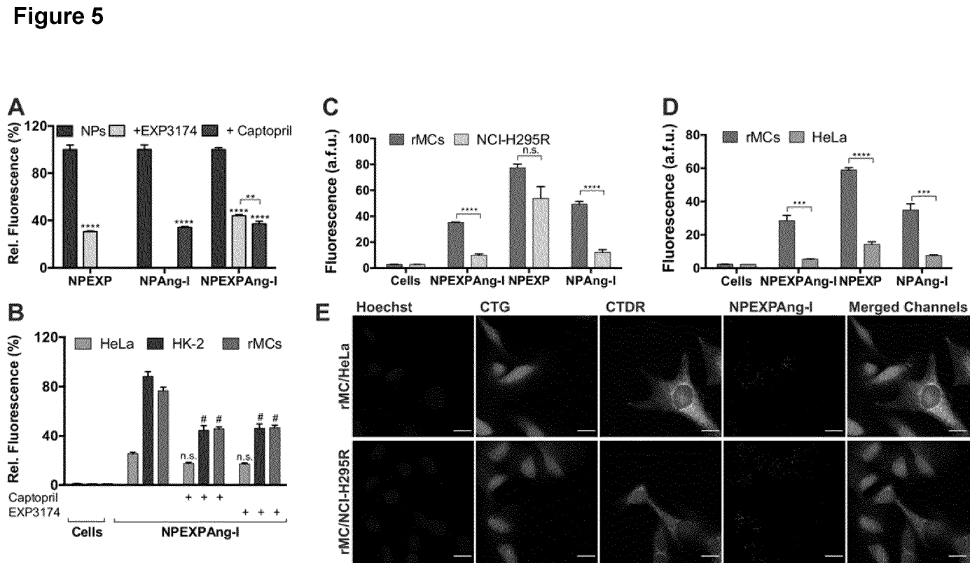

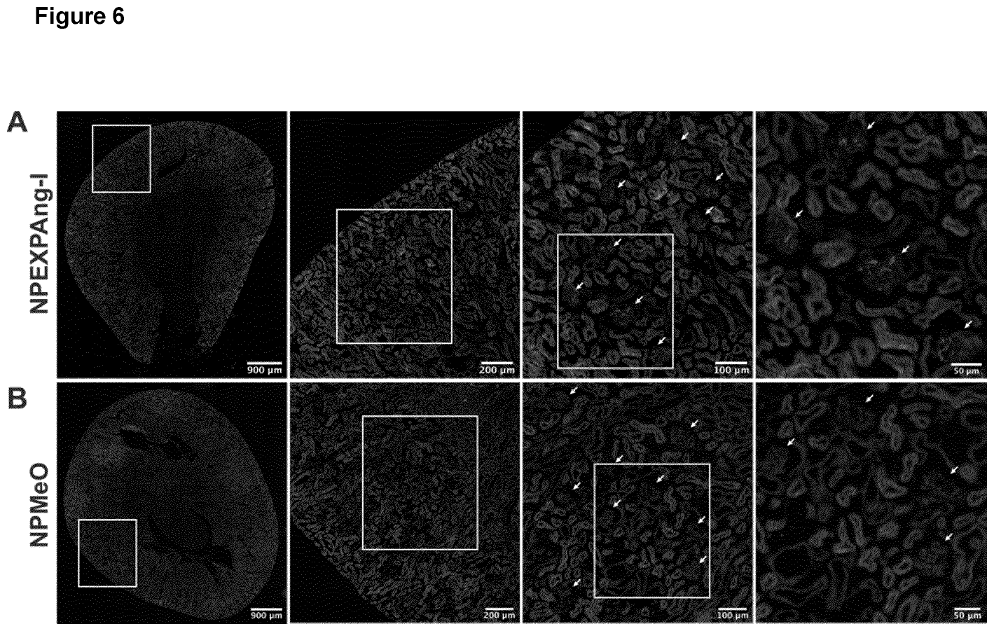

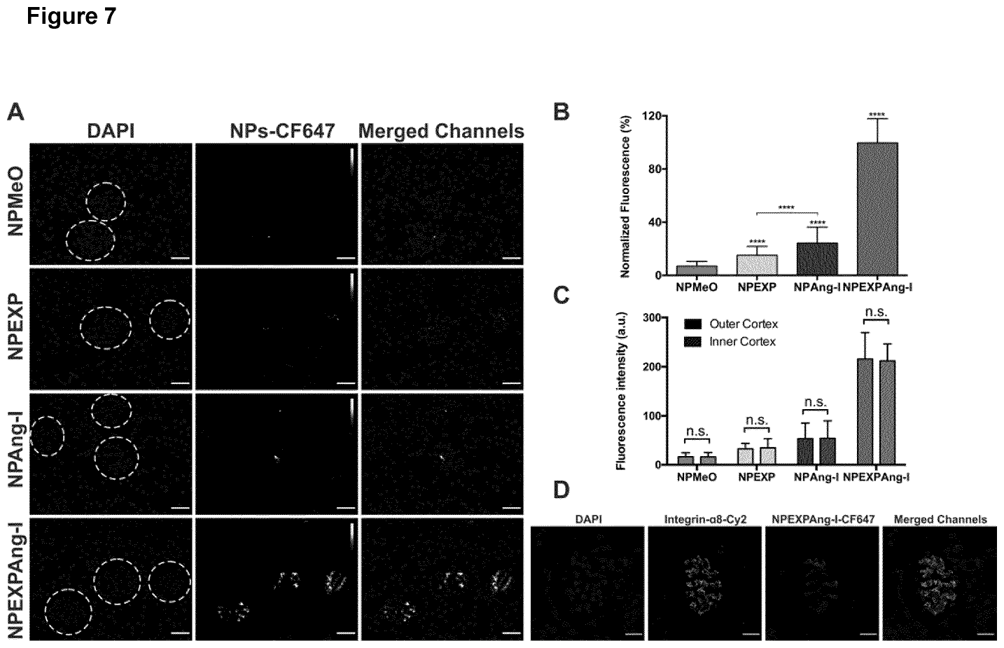

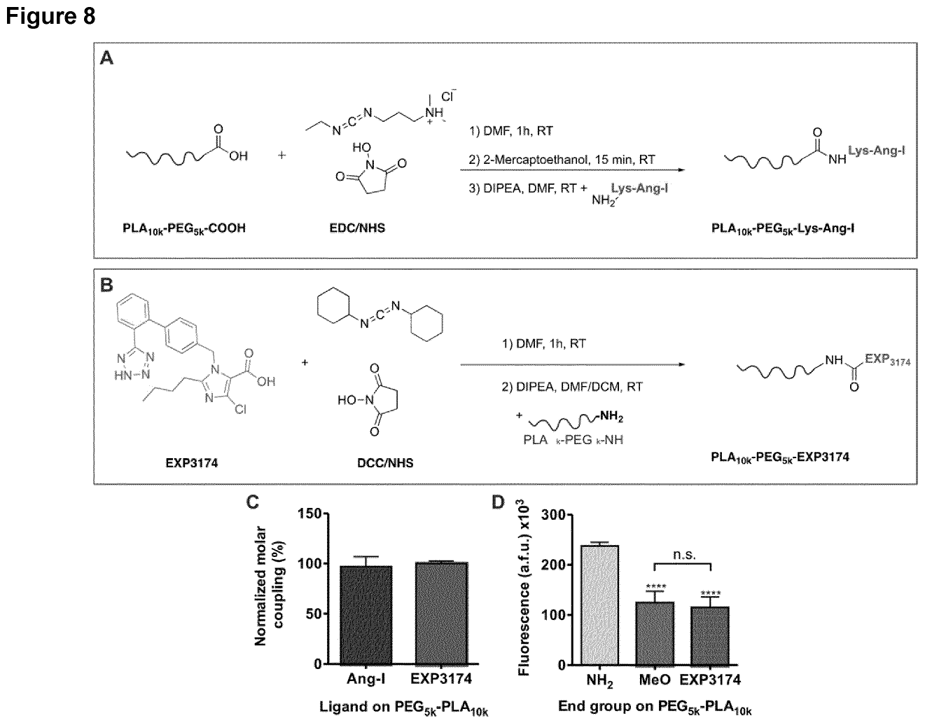

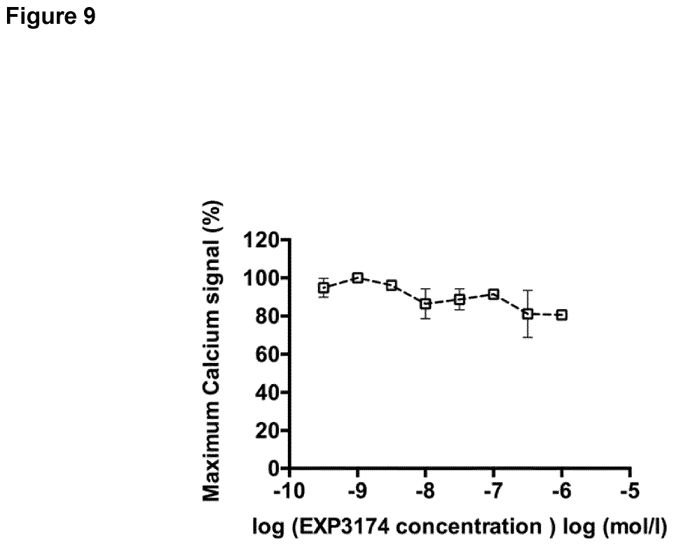

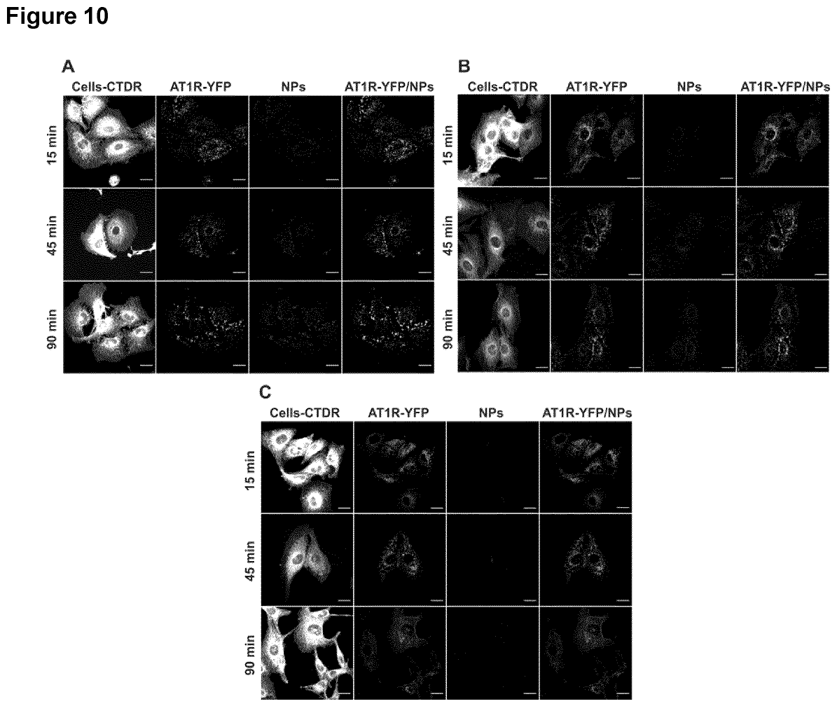

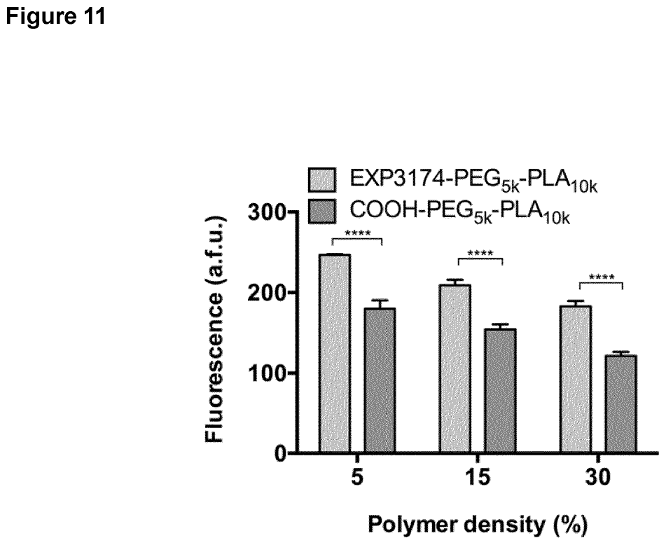

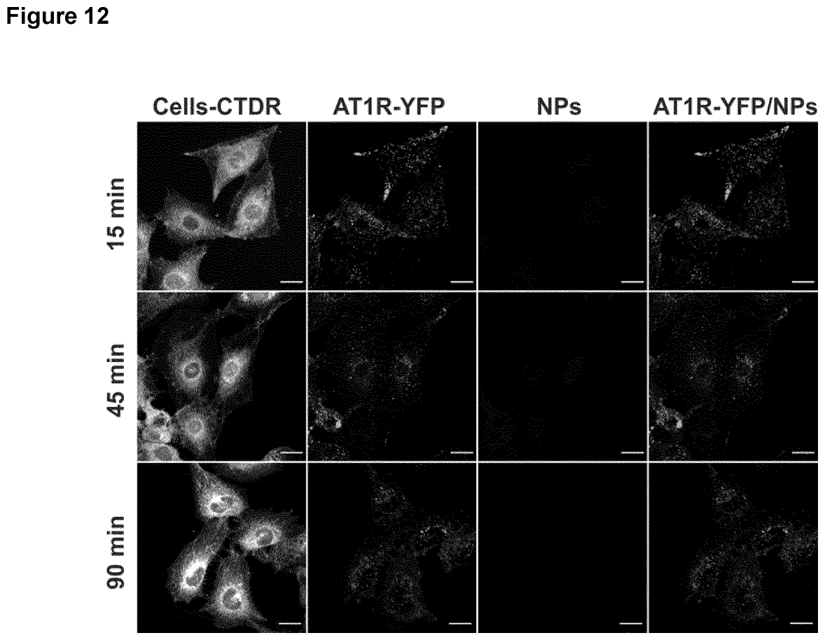

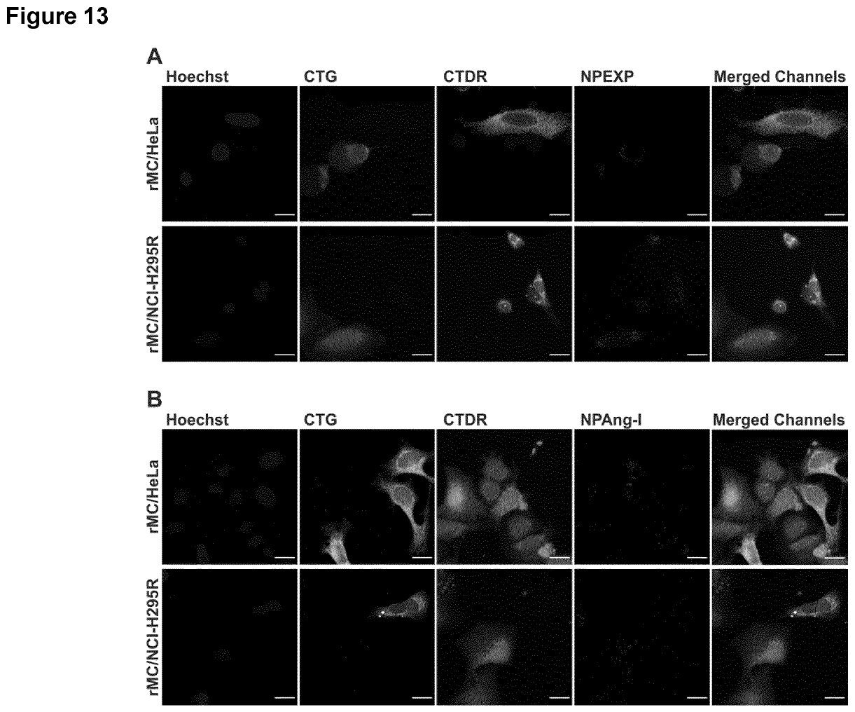

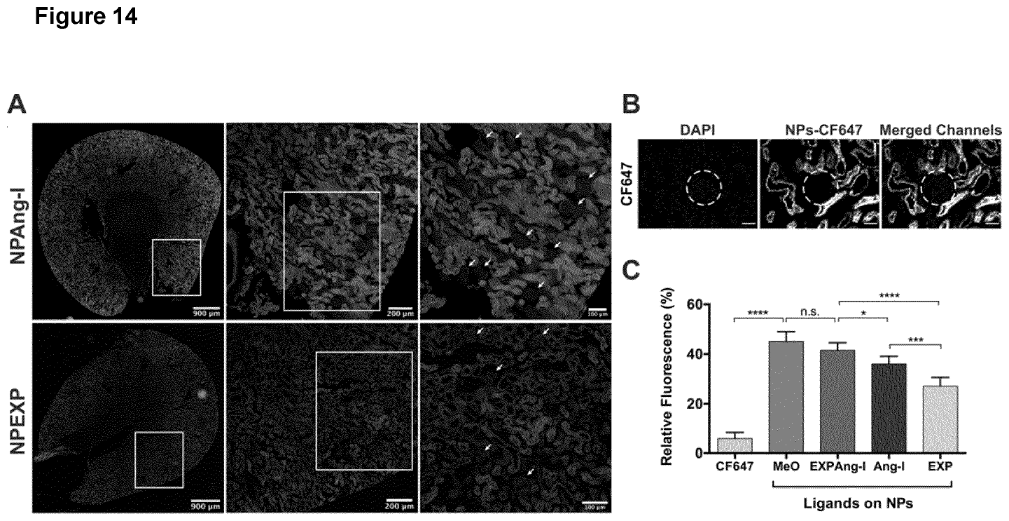

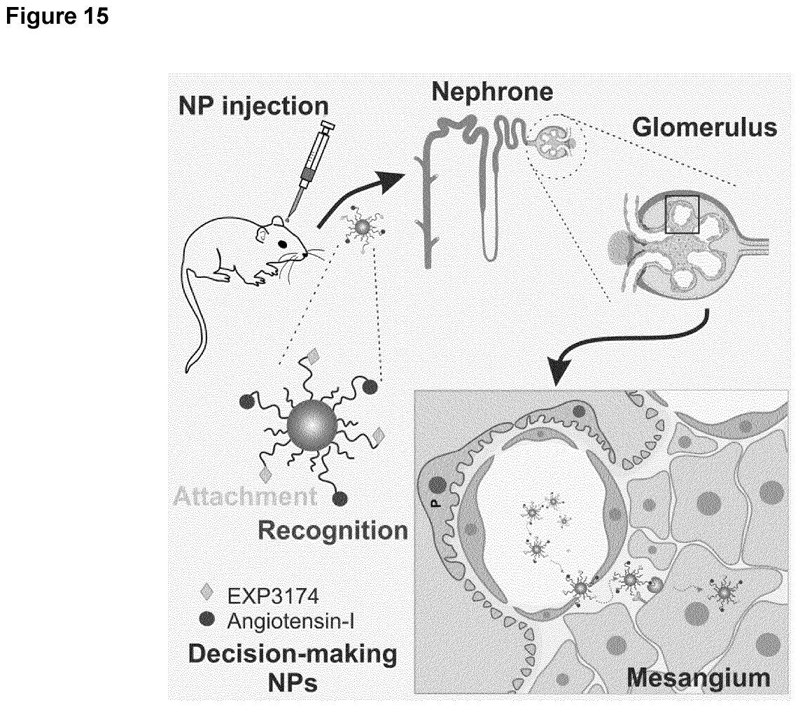

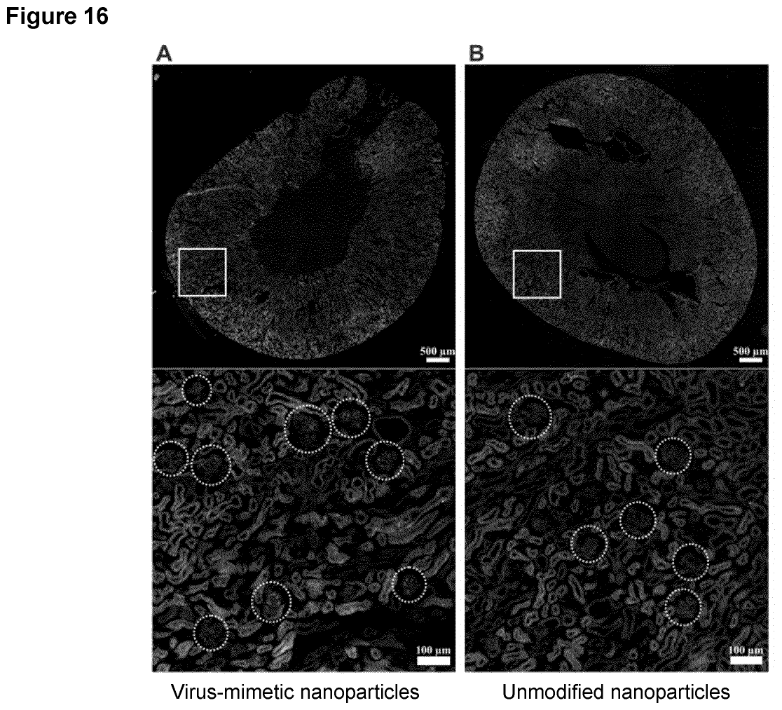

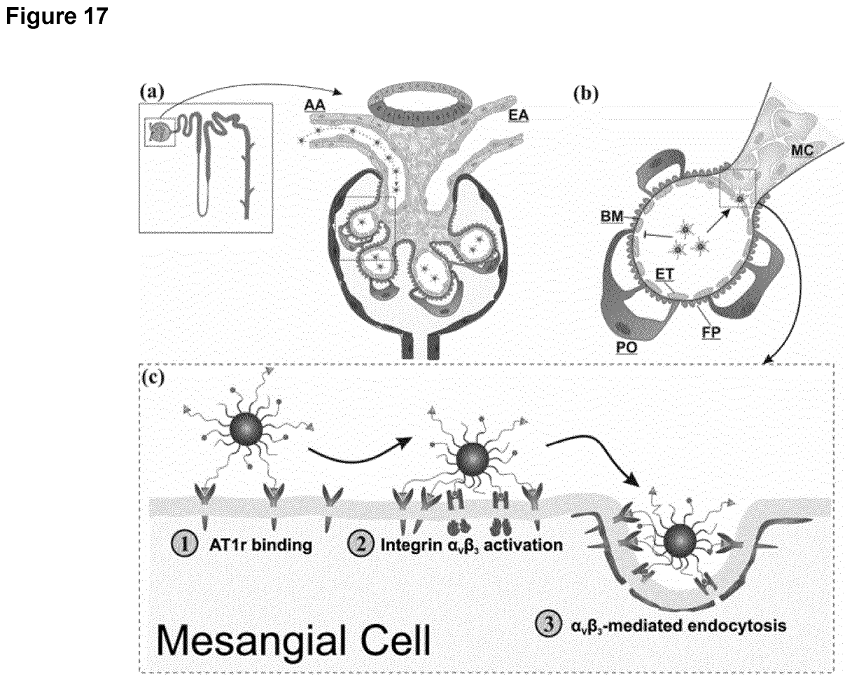

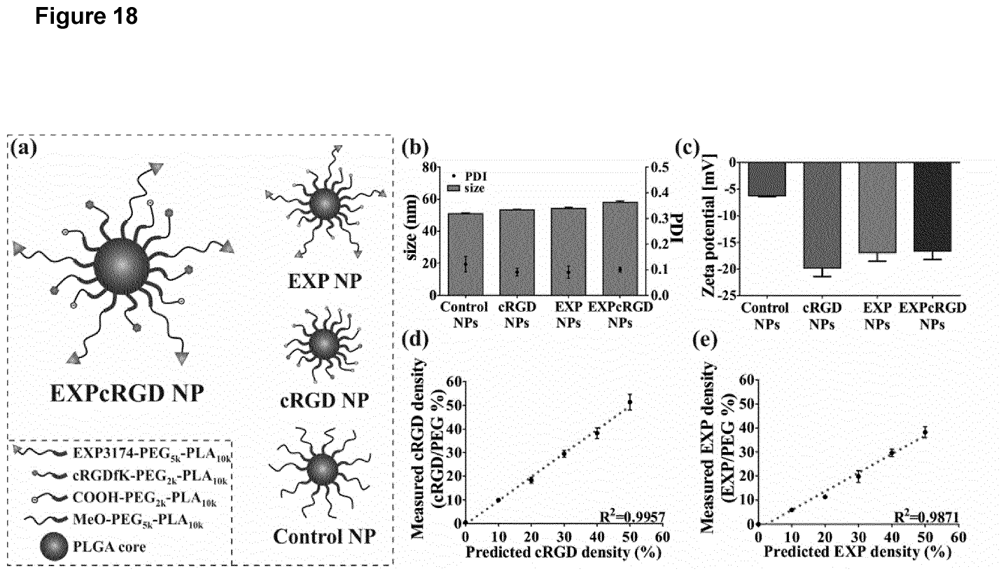

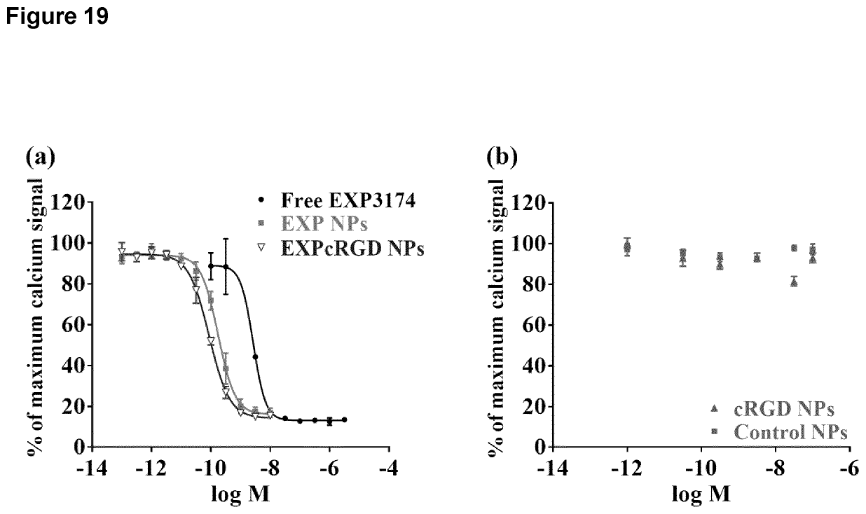

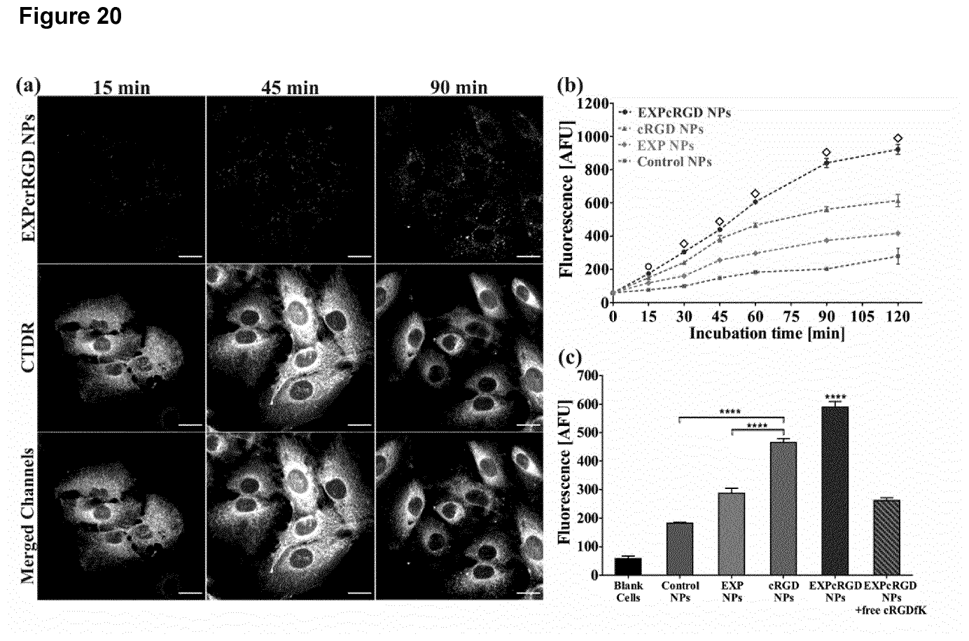

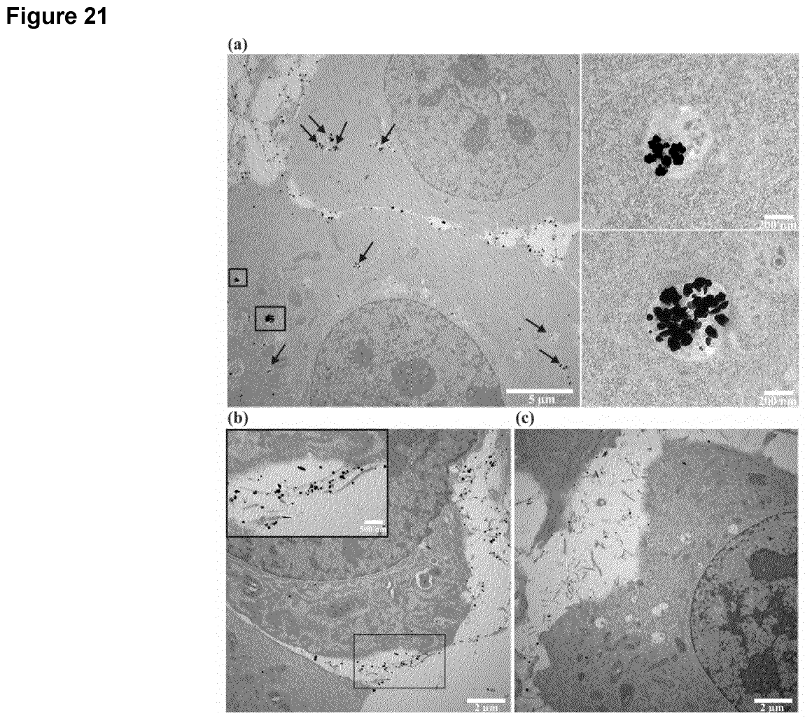

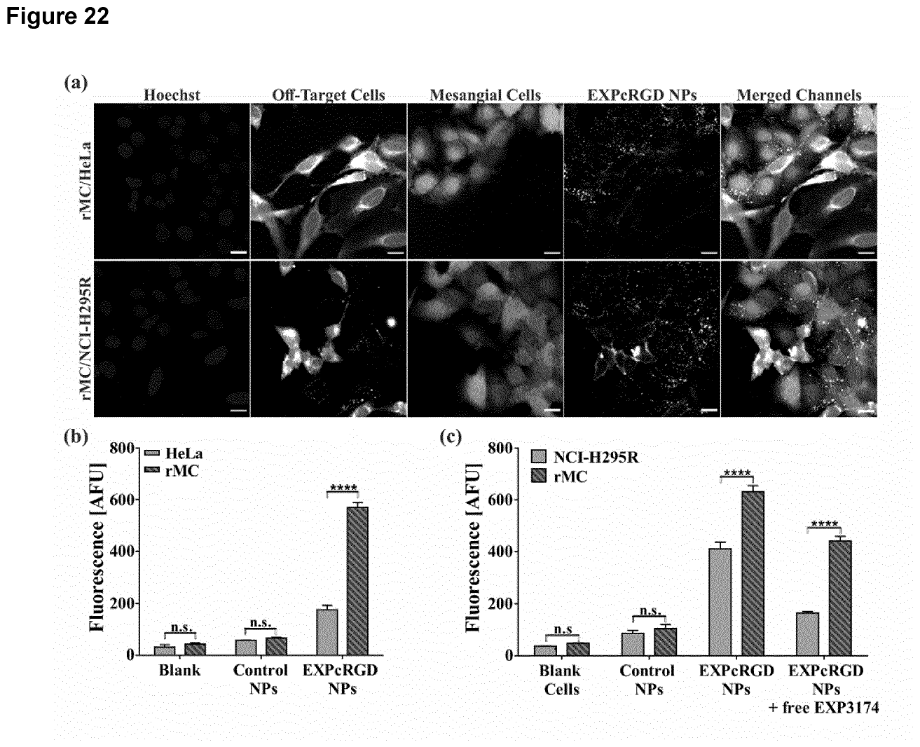

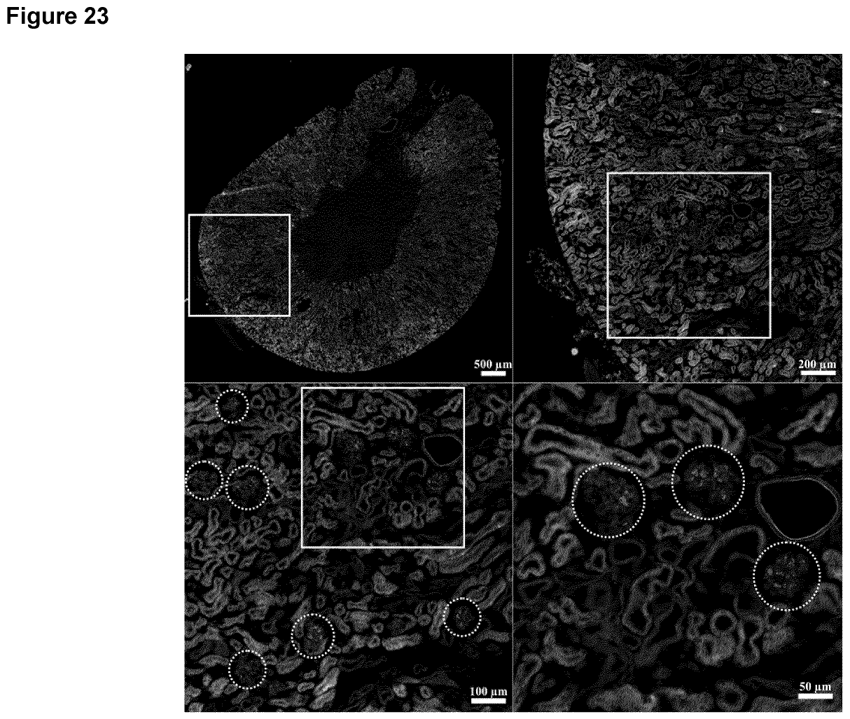

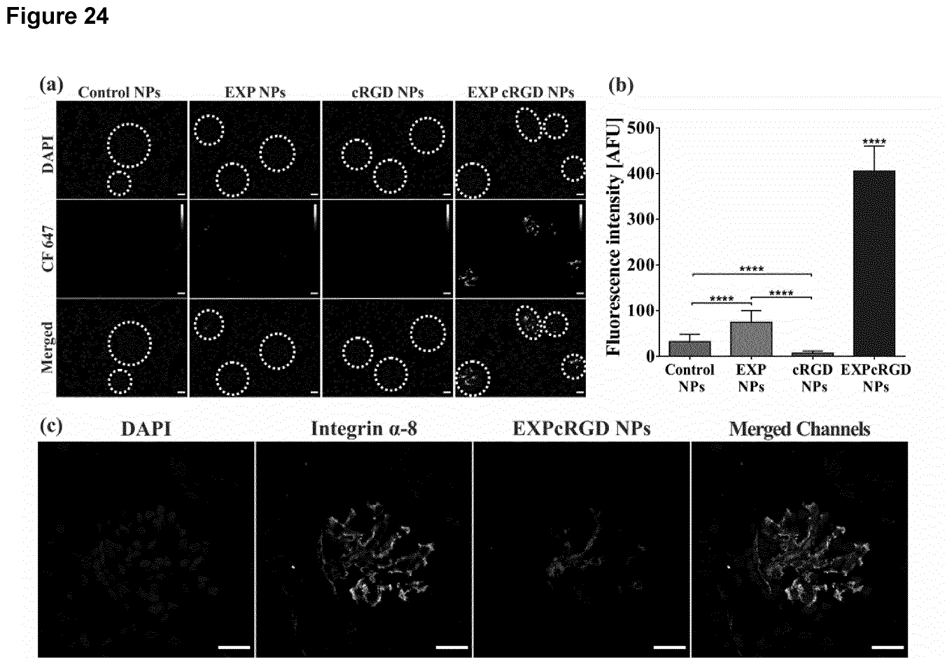

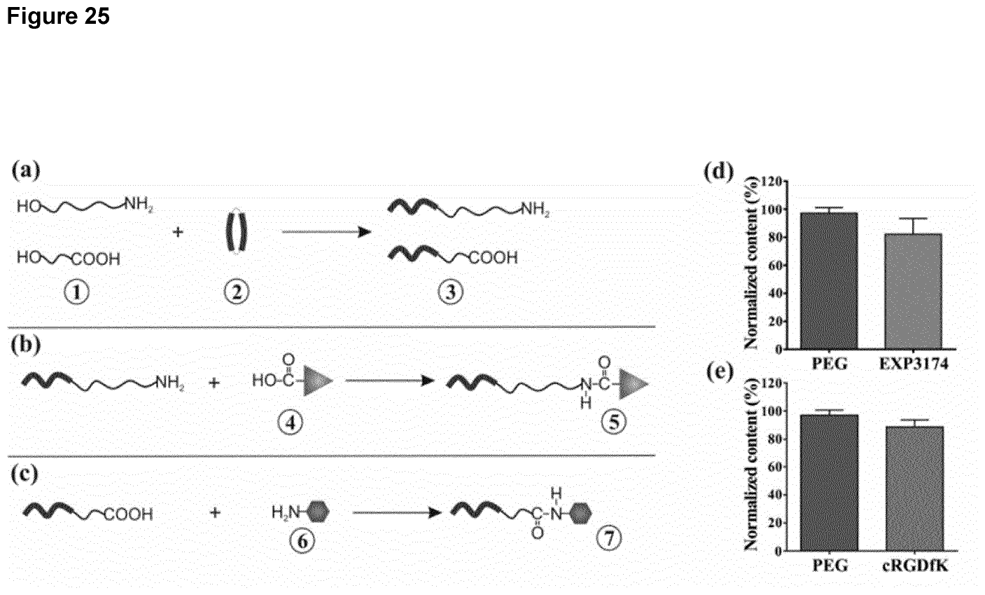

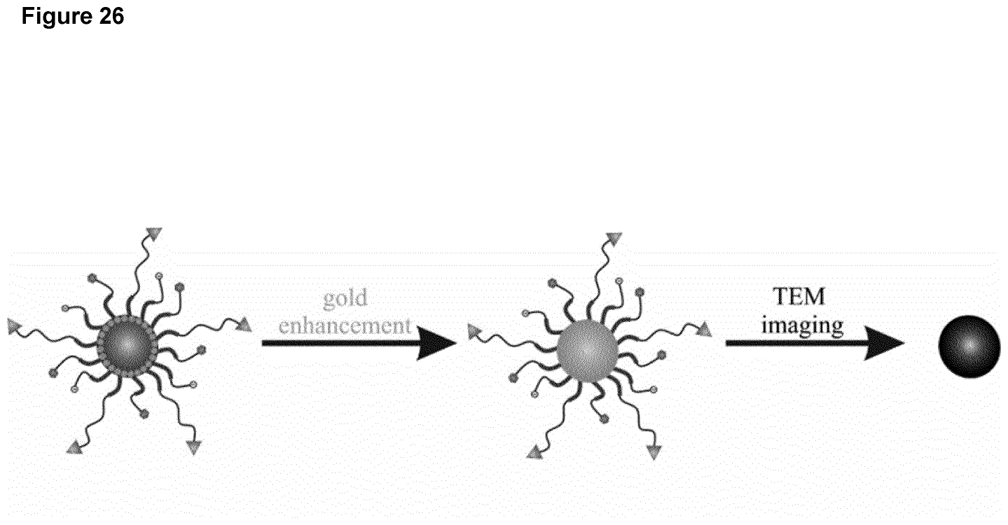

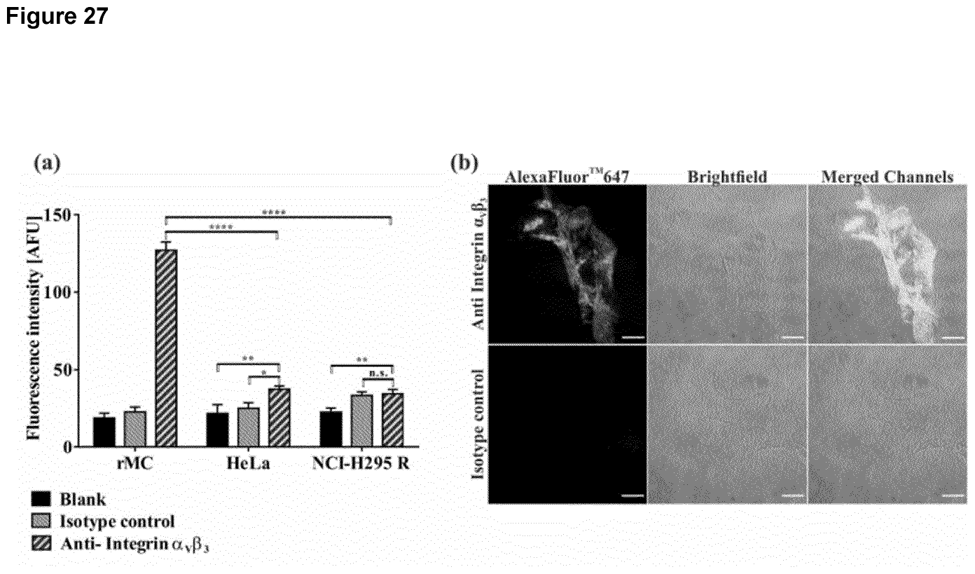

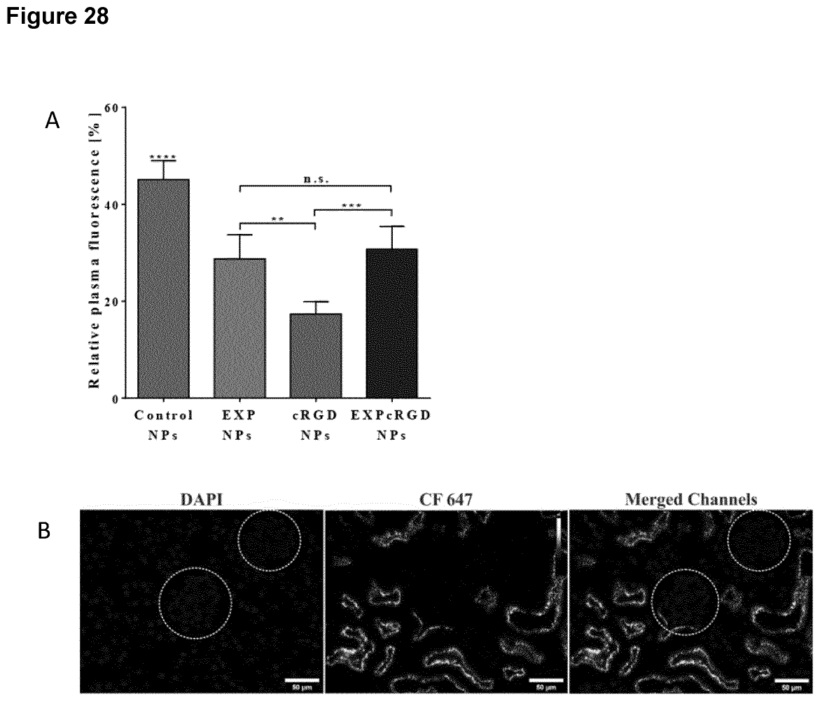

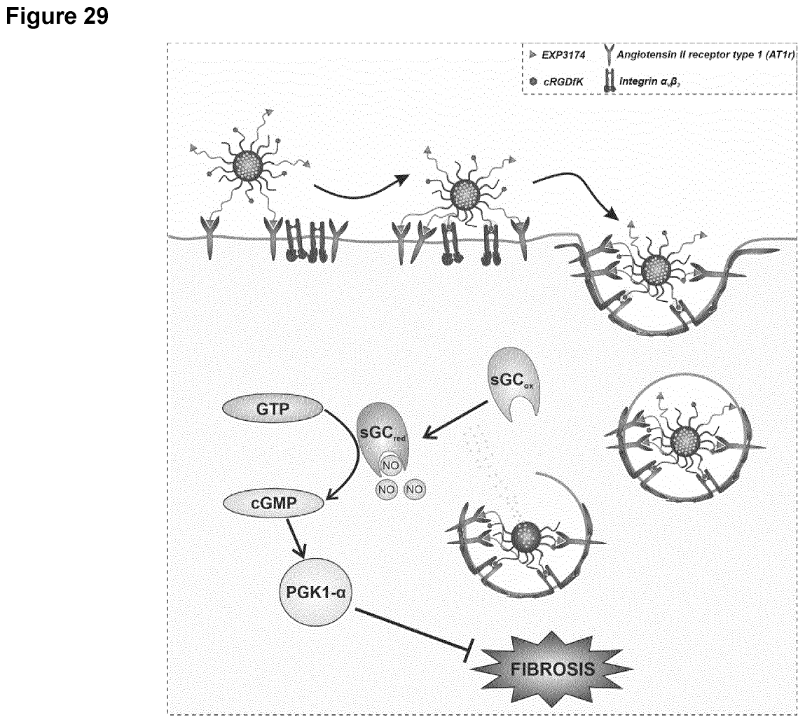

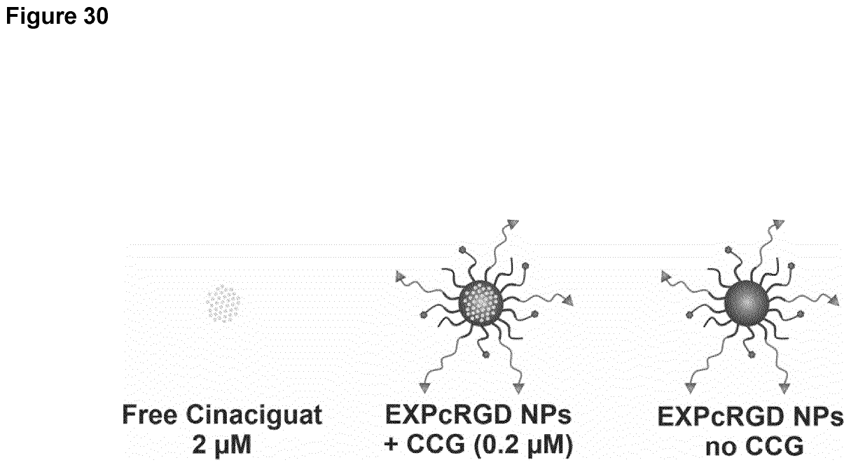

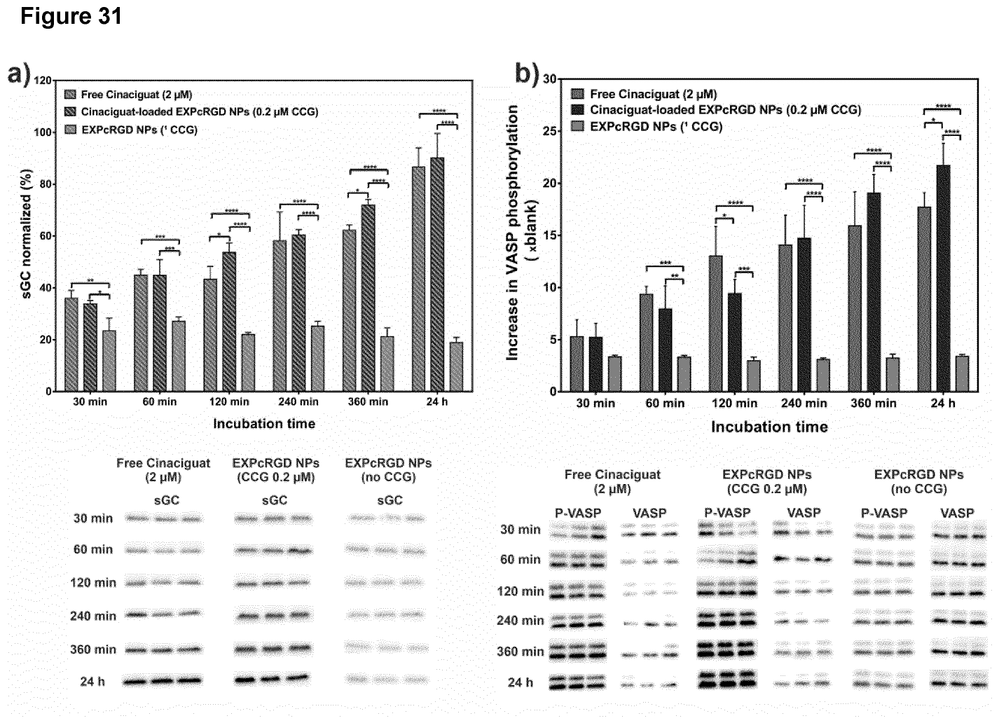

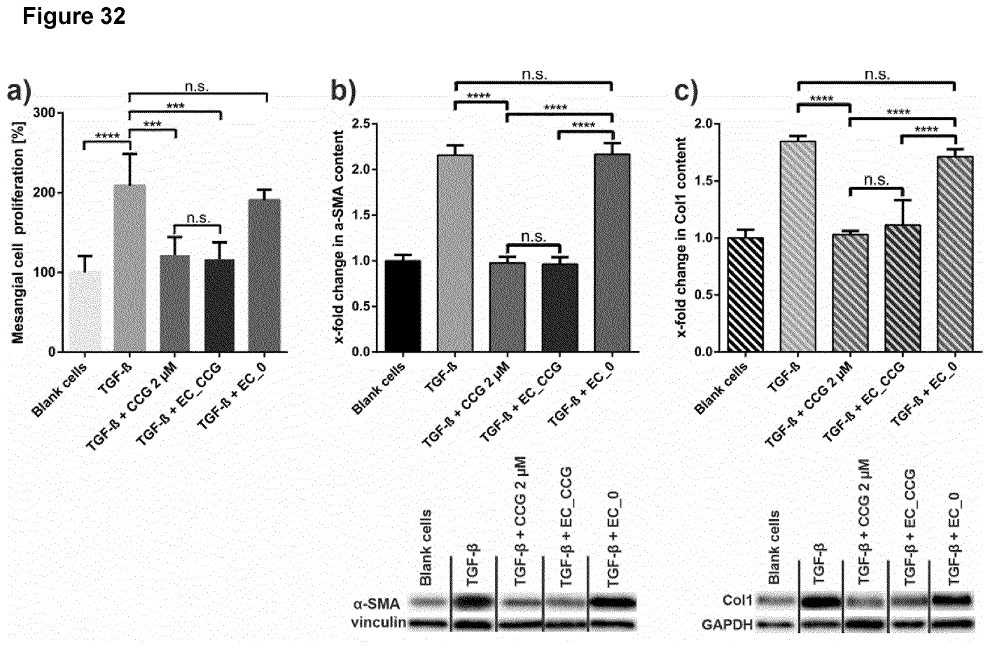

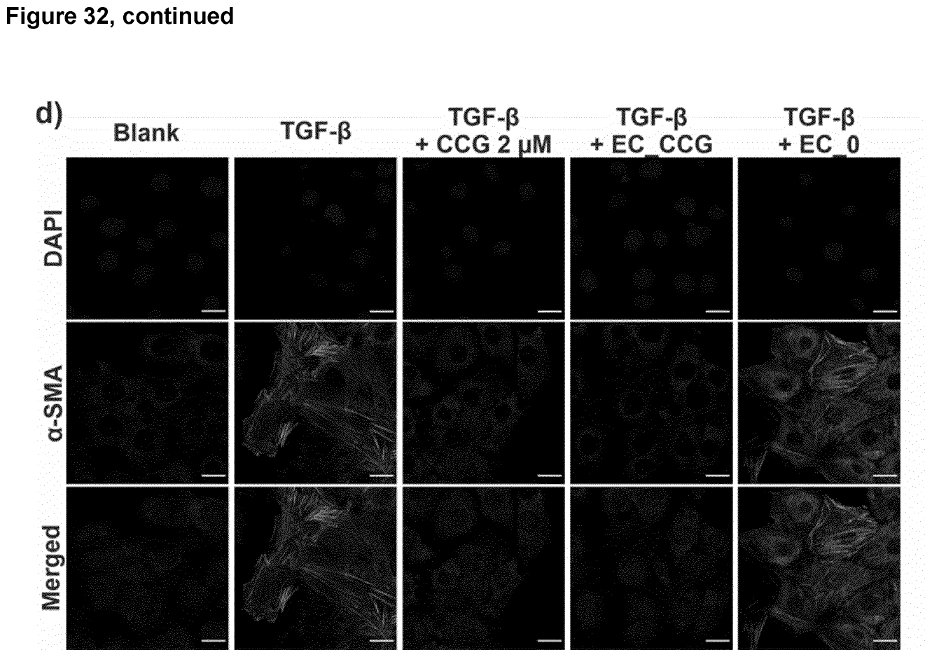

In order to assess the AT1R interaction of the different NP formulations a ratiometric Fura-2 Ca²⁺ chelator method was used as previously described [1, 3] using AT1R positive rMCs. For that purpose, rMCs were seeded in T-150 flasks (Corning) and incubated until confluent. Subsequently, they were trypsinized, centrifuged (200g, 5 min) and resuspended in Leibovitz medium supplemented with 5 μM Fura-2, AM (Thermo Fisher), 0.05% Pluronic F-127, and 2.5 mM Probenecid. Cells were incubated for 1 hour, light protected, with gentle agitation (50 rpm). Afterwards, the cell suspension was washed with DPBS by centrifugation (2×, 200 g, 5 min, RT), and resuspended in Leibovitz medium supplemented with 2.5 mM Probenecid at a count of 2 million cells/mL. To determine the particle avidity and ligand affinity for the AT1R (FIGS. 3A-C) 45 μl of Fura-2-loaded-rMC in suspension (90,000 cells/well) were incubated with 10 μL of different samples (NPs or free ligands ranging from 1 nM to 300 μM (ligand concentration)) at r.t. for 30 minutes in a 96-well half-area microplate. Afterwards, cells were stimulated with 45 μL of a 300 nM Lys-Ang-II aqueous solution and the resulting calcium signal immediately recorded for 1 min/well using a FLUOstar Omega microplate reader (BMG Labtech) with 340/20 nm and 380/20 nm excitation and 510/20 nm emission bandpass filters. To determine the kinetics of the AT1R interaction (FIG. 3D) the same procedure was used, but the samples were incubated for different time periods (from 5 to 320min) with the cells. The maximal and minimal signal ratio, was determined by stimulating the cells with 0.1% Triton-X 100 or 0.1% Triton-X 100 with 45 mM ethylene glycol-bis(2-aminoethylether)-N,N,N′,N′-tetraacetic acid (EGTA), respectively. The intracellular calcium concentrations were calculated after Grynkiewicz assuming a K_{d} value of 225 nM. Statistical significance was assessed through a Student's t-test (FIG. 3C) and a 2-way ANOVA using Sidak's multiple comparisons (FIG. 3D) using GraphPad Prism 6.0.

Enzyme Kinetic Measurements

The Michaelis-Menten kinetics for NPAng-I and NPEXPAng-I were determined as previously described [1] using rabbit lung ACE (Sigma Aldrich) as a soluble surrogate for the cell membrane-bound enzyme. In short, different concentrations of NPs (corresponding to 10-120 μM Ang-I) were incubated with 18 μM of enzyme for different time periods (5, 15, 30, 60, 90 and 120 min) to convert the Ang-I on the particle corona to the AT1R-active ligand Ang-II. The resulting Ang-II was quantified by direct intracellular calcium measurements. To that end, rMCs were loaded with Fura-2 dye as described above. Then, 10 μL samples were pipetted onto a 96-well half area plate and 90 μL of a Fura-2-loaded rMC-suspension (90,000 cells/well) were injected on top of them. The resulting calcium signal was recorded immediately over 1 minute using a FLUOstar Omega microplate reader (BMG, Labtech), as described above. In order to rule out the interference of the EXP3174 ligand on NPEXPAng-I under the experimental conditions used, NPEXP were used as a control (see FIG. 9). rMCs (90,000 cells/well) were simultaneously stimulated with different NPEXP concentrations and Lys-Ang-II (400 nM) and the resulting calcium signal recorded immediately over 1 minute. Free Lys-Ang-II in known concentrations ranging from 1 nM to 300 μM were used to convert the measured calcium concentration to pmol of hydrolyzed product. The velocity of the reaction (pmol/min) at 15 minutes incubation time was plotted against the substrate concentration used in the assay to determine the Michaelis-Menten constant (Km) for particle- and ligand based-concentrations (FIG. 3E) using GraphPad Prism 6.0. The catalytic constant (Kcat) was obtained using the same software in order to calculate the specificity constant (Kcat/Km) used to compare different substrates for the same enzyme (FIG. 3F). Statistical significance (FIG. 3F) was assessed though a 2-way ANOVA with Tukey's multiple comparison test using GraphPad Prism 6.0.

Cellular Distribution of NPs: Confocal Microscopy

In order to determine the cellular distribution of the different particle formulations (FIG. 4), pAT1R-rMCs were seeded into 8-well μ-slides (Ibidi, Graefelfing, Germany) at a density of 10,000 cells/well and incubated over 24 hours (37° C.). Then they were incubated with pre-warmed NP-solutions (0.2 mg/ml) in Leibovitz medium (LM) supplemented with 0.1% bovine serum albumin (BSA) for 15, 45 or 90 minutes. Afterwards, the NPs were discarded, and the cells washed thoroughly with DPBS prior to cell staining with 1× CellMask™ Deep Red Plasma Membrane Stain for 5 minutes and fixation with 4% paraformaldehyde (PFA) in DPBS for 10 minutes at r.t. Images were acquired using a Zeiss LSM 700 microscope with the focal plane set at 1.4 μm using the Zen Software (Carl Zeiss Microscopy). For particle uptake and binding inhibition cells were preincubated with 1 mM free EXP3174 or captopril prior to particle addition. Images were analyzed using Fiji software.

Cellular Distribution of NPs: Flow Cytometry

Analysis of particle uptake through flow cytometry (FIG. 5A) was performed as previously described [1]. In short, rMCs were seeded in 24-well plates at a density of 30,000 cells/well and incubated for 48 h (37° C.). Prewarmed NP-solutions (0.7 mg/ml in LM supplemented with 0.1% BSA) were pipetted on top of the cells, after washing them with DPBS, and incubated for 45 min at 37° C. To confirm the uptake specificity, cells were incubated with 1 mM of captopril and/or EXP3174 for 30 min prior to particle addition. Afterwards, particle solutions were discarded, and the cells washed thoroughly with DPBS, trypsinized and centrifuged (2×, 200 g 5 min, 4° C.). NP-associated cell fluorescence was analyzed in DPBS using a FACS Calibur cytometer (Becton Dickinson). Fluorescence was excited at 633 nm and recorded using a 661/16 nm bandpass filter. The population of viable cells was gated using Flowing software 2.5.1. (Turku Centre for Biotechnology) and the geometric mean of the NP-associated fluorescence was analyzed. Statistical significance (FIG. 5A) was assessed through a Student's t-test using GraphPad Prism 6.0.

NP Target Cell Specificity: Flow Cytometry

To assess the NP uptake in different cells lines (FIG. 5B) rMCs, HK-2 and HeLa cells were seeded out in 24-well plates at a density of 30,000, 50,000 or 100,000 cells/well, respectively and incubated over 48 h (37° C.). Afterwards pre-warmed NP-solutions (0.7 mg/ml in LM supplemented with 0.1% BSA) were added on top of the cells and processed as described above. Statistical significance (FIG. 5B) was assessed through a 2-way ANOVA with Sidak's multiple comparisons test using GraphPad Prism 6.0.

The particle specificity in co-culture of target and off-target cells was investigated through flow cytometry (FIG. 5C-D) as previously described [1]. Briefly, CTG-stained rMCs (10 μM in serum-free medium, 30 min, 37° C.) were seeded in 24-well plates in co-culture with unstained off-target NCI-H295R or HeLa cells at densities of 10,000 and 75,000 cells/well, respectively, and incubated for 48 h. Warm NP solutions at concentrations of 0.02 mg/ml in LM supplemented with 0.1% BSA were subsequently added on top of the cells and incubated for 45 min at 37° C. Afterwards, particles were discarded, and cells processed for flow cytometry analysis as described above. Statistical significance was assessed through a Student's t-test using GraphPad Prism 6.0.

NP Target Cell Specificity: Confocal Microscopy

To confirm the flow cytometry experiments, CLSM analysis of the particle specificity in co-culture (FIG. 5E and FIG. 13) was performed as previously described [1]. In short, target CTG-stained rMCs (10 μM, 30 min, 37° C.) were seeded in co-culture with CTDR-stained off-target HeLa or NCI-H295R cells (25 μM, 30 min, 37° C.) at densities of 2,000 and 10,000 cells/well, respectively, and incubated for 24 hours. Afterwards, the cell nuclei were stained for 20 minutes with Hoechst 33258 (5 μg/ml in DPBS) and the prewarmed 0.02 mg/mL NP solutions in LM supplemented with 0.1% BSA were pipetted on top of the cells and incubated for 45 minutes. Then, the NP solutions were discarded, and the cells washed thoroughly with DPBS and fixated for 10 min with 4% PFA in DPBS (r.t.). Images were acquired and analyzed using a Zeiss LSM 700 microscope and Fiji software, respectively, as described above.

NP Kidney Distribution In Vivo

To assess the kidney distribution of the different NP formulations (NPEXPAng-I, NPAng-I, NPEXP and NPMeO), 100 μL of a 120 nM NP solution (equivalent to approximatively 10 mg/ml NPs) were injected via the vena jugularis in 10-week old female NMRI mice (Charles River) that were anesthetized with isoflurane inhalation and buprenorphine (0.1 mg/kg body weight) (n=6 for each particle sample). Additionally, as a control 100 μL of the free dye used to fluorescently label the particles (CF647) was injected in the same concentration contained in a particle sample (approximatively 50 μM). After 5 min a blood sample was collected via i.v. punction while mice were still under anesthesia. After 1 h of particle circulation mice were anaesthetized with ketamine/xylazine, a final blood sample was collected and they were killed through perfusion fixation with 4% PFA. The kidneys were harvested and cut transversally. They were cryoprotected by placing them in phosphate buffer (0.1 M pH 7.4) supplemented with 18% sucrose and 1% PFA overnight. Afterwards, they were frozen in liquid nitrogen-cooled 2-propanol (−40° C.) and embedded in Tissue Tek® O.C.T.™ Compound for cryosections. Kidneys were cut into 5 μm sections using a CryoStar NX70 cryostat (Thermo Fisher Scientific) and transferred onto Superfrost™ plus glass slides. For better visualization cell nuclei were stained with DAPI (12.5 μg/ml in DPBS) prior to section imaging using an Axiovert 200M (Zeiss) fluorescence microscope and Zen software (Zeiss). Images of the whole kidney were acquired using a lox objective (FIG. 6). For glomerular fluorescence quantification images were taken using a 40× objective (an average of 120 glomeruli per sample) and analyzed using Fiji Software (Schneider et al., 2012). For better visualization the lookup table “Red Hot” was applied to the particle-associated fluorescence. The area of each glomerulus was quantified, and the fluorescent area gated. Then, the integrated fluorescence density of each gated area was quantified and correlated to the whole glomerulus area. Statistical significance was assessed through a Student's t-test using GraphPad Prism 6.0. In order to compare the particle-associated fluorescence of the inner and outer cortex, the cortex was divided into two equal sections and the glomerular fluorescence analyzed as described above. To assess statistical significance a 2-way ANOVA with Sidak's multiple comparisons test was performed using GraphPad Prism 6.0. NP-associated fluorescence in plasma was measured using a FLUOstar Omega microplate reader (BMG Labtech) with excitation and emission wavelengths of 640 and 680 nm, respectively. Fluorescence ih after injection was correlated to the initial fluorescence of the sample obtained 5 min after injection.

Immunohistochemistry

To assess the glomerular localization of NPs, freshly cut 5 μm kidney cryosections were washed for 5 min with DPBS, 5 min with DPBS supplemented with 0.1% sodium dodecyl sulfate (SDS) and 5 min with DPBS prior to 10 min-blockage with 5% BSA in DPBS supplemented with 0.04% Triton-X (m/v). Sections were washed again with DPBS (5 min) and incubated overnight in a 1:200 solution of the primary polyclonal goat anti-Integrin-α8 antibody in DPBS supplemented with 0.5% BSA and 0.004 Triton-X (m/v) at 4° C. Then, they were washed for 5 minutes in DPBS and incubated for 1 h with the Cyt-anti-goat secondary antibody (1:400) and DAPI (12.5 μg/ml) in DPBS supplemented with 0.5% BSA and 0.04% Triton-X at r.t. in the dark. Cryosections were washed with DPBS and ultrapure water before they were mounted using Dako Faramount Mounting Medium and analyzed using a Zeiss LSM 700 microscope and Fiji software, as described above.

Quantification and statistical analysis

Statistical analysis was performed using GraphPad Prism Software 6.0. Student t tests or two-way ANOVA with a Sidak's or Turkey's multiple comparison test were employed to evaluate statistical significance as indicated in the method details. Levels of statistical significance and “n” numbers for each experiment are indicated in the text and figure legends.

### Example 2: Block Copolymers Allow for a Virus-Mimetic Particle Design

All materials and methods mentioned in this example were as described in the previous example.

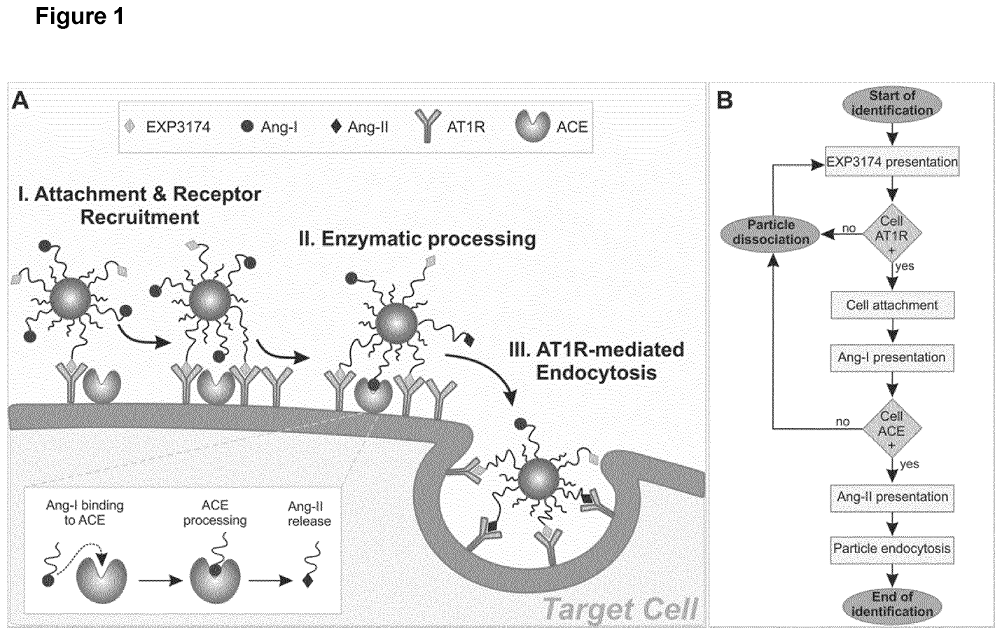

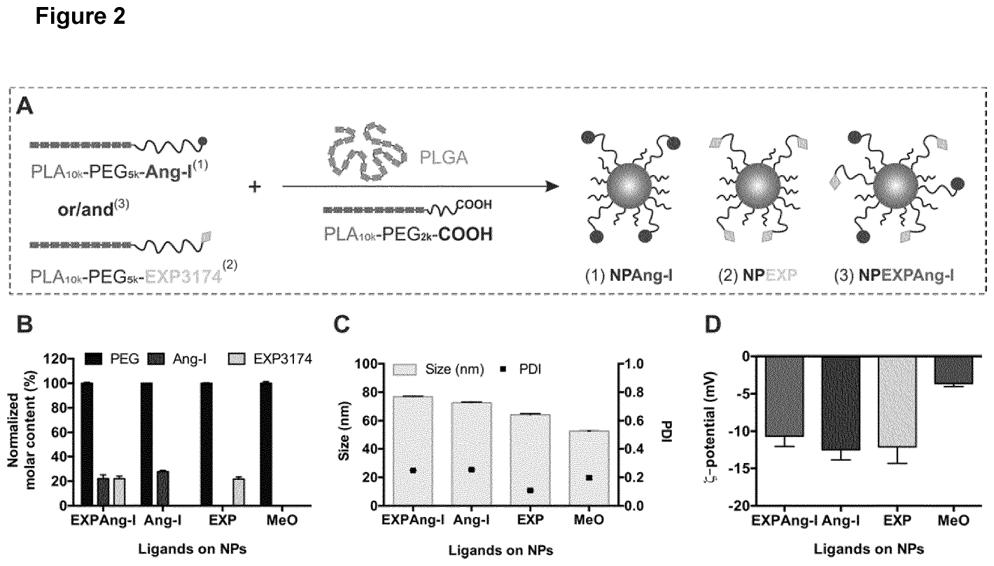

For the development of virus-mimetic NPs, the present inventors coupled the ligands EXP3174 and Ang-I to poly(ethylene glycol)5k-poly(lactic acid)10k (PEG-PLA) block copolymers (FIG. 8), which were blended with poly(lactic-co-glycolic acid) PLGA for NP manufacturing via bulk nanoprecipitation rendering particles with sufficient stability in vivo. The remaining, non-functionalized polymers were carboxylic acid-ended PEG-PLA with a shorter 2 k PEG and a ink PLA block (COOH-PEG2k-PLA10k) (FIG. 1A). By modifying the polymers with ligands prior to NP preparation, an exact control over the ligand density can be obtained. Particles were prepared such that 20% of their PEG chains were decorated with Ang-I and an additional 20% with EXP3174 (NPEXPAng-I) (FIG. 2B). The ligand density was kept at a 40% maximum to avoid stearic hindrance among ligands and non-specific interactions. As a control, ligand-free methoxy-PEG-terminated particles (NPMeO) and particles carrying either 20% Ang-I or EXP (NPAng-I and NPEXP, respectively) were assembled (FIG. 1A). By combining long ligand-carrying polymers with shorter non-functionalized polymers for particle preparation, the size of the NPs could be kept under 80nm to endow particles with the ability of passing through the endothelial fenestrations of mesangial capillaries (FIG. 2C). Carboxylic acid terminated block copolymers were selected as a filler that provides an overall negative particle charge ideal to avoid non-specific electrostatic adsorption to the negative cell membranes (FIG. 2D).

### Example 3: NPs Recognize Target Receptors In Vitro

All materials and methods mentioned in this example were as described in the previous examples.

To confirm the particles' ability to triple check a cell's identity was initially assessed in vitro. Particle avidity for the target receptor, which mediates primary attachment and subsequent internalization, was investigated using calcium mobilization assays, since the stimulation or silencing of the Gq-coupled AT1R with an agonist or antagonist results in a cytosolic Ca²⁺ influx or its suppression, respectively. To that end, ATTR-positive rat mesangial cells (rMCs) were incubated for a 30 minute period with varying concentrations of either free ligands or NP-formulations prior to stimulation with Ang-II and recording the resulting calcium signal. As depicted in FIG. 3A, control experiments with free EXP3174 and Ang-II revealed a high affinity of both compounds for the AT1R in the nanomolar range (IC50 values of 0.6±0.4 and 1.5±0.1 nM, respectively). Ang-I displays a lower affinity (IC50 0.9±0.6 μM), as the receptor binding and activation occurs only after enzymatic conversion to Ang-II by ACE present in the cell membrane.

The coupling to linkers leads to an affinity loss that is compensated by the high avidity multivalent binding of several receptors simultaneously (FIGS. 3B and 3C). Particles that only carry Ang-I (NPAng-I) show a lower avidity for the AT1R (IC50 of 9.4±0.4 nM) than particles carrying EXP3174 (IC50 of 0.4±0.1 nM), as their primary interaction is with the ACE. Nevertheless, particle binding of Ang-I leads to a significant decrease in IC₅₀ values compared to the free ligand due to a facilitated enzymatic cleavage at the NP-interface and the subsequent multivalent binding. EXP3174-modified NPs in contrast, had avidities that were of the same order of magnitude as for the free ligand. Surprisingly, particles that carried both ligands, NPEXPAng-I, showed a cooperative effect with respect to receptor binding, as they had significantly higher avidity for the AT1R (IC₅₀ of 0.2±0.09 nM) than either of the particles carrying only one type of ligand (FIG. 3C). This proves that the ligands do not hinder each other's interaction, as after Ang-I enzymatic activation to Ang-II both ligands target the same receptor in a simultaneous agonistic and antagonistic manner. Particles without any functionalization (NPMeO) confirmed that the assay was ligand-specific, as they did not elicit any response (FIG. 3B).

To assess kinetics of cell/particle interactions, intracellular calcium measurements were performed over a 5.5-hour period by incubating NPs with rMCs at a concentration corresponding to 10 μM ligand. The extent to which they could silence calcium signaling triggered by the present free agonist served as a measure for the completeness to which the respective particles had bound via their ligands to the AT1R in the cell surface at different time points (FIG. 3D). Particles carrying only Ang-I on their surface displayed a slow receptor binding since they initially need to be activated by the cell membrane-bound ACE to Ang-II carrying particles before they can interact with the AT1R. The receptor binding reached a maximum at about 40% after i-hour incubation, which remained constant over the assay's duration. This points towards a fast internalization of the particles once a certain number of proligand is activated, with possibly not all Ang-I being converted to Ang-II. Once Ang-II on the particle surface binds to a receptor, the particles are rapidly internalized (as they have picomolar AT1R avidities [1]) which means that not all proligands may need to be activated for NP internalization to occur. This phenomenon is avoided when adding EXP3174 as an attachment factor on the particle surface. A very fast and complete receptor blockage occurs after only 5 minutes of particle incubation (for NPEXPAng-I and NPEXP alike). The AT1R inhibition is maintained over almost the whole measurement and descends to about 80% at the last time points, probably due to receptor upregulation and recycling. The attachment by EXP3174 to the cell membrane slows down the recognition process and enables a higher Ang-I to Ang-II activation that can more efficiently bind to the AT1R. Comparing NPEXPAng-I and NPEXP there is a significantly higher initial AT1R inhibition of NPEXPAng-I which evens out after 45 minutes of particle incubation. This is likely due to the combined effect of the two ligands which leads to a higher avidity for the AT1R (FIG. 3C).

A prerequisite for particle internalization is the ability of ACE to activate Ang-I to Ang-II. Therefore, the present inventors investigated the enzyme kinetics for NPEXPAng-I, to determine whether the presence of the antagonist on the particle surface would hinder the enzymatic reaction. A soluble form of ACE was incubated for varying time periods with different particle concentrations and the resulting Ang-II on the NP corona was quantified running calcium mobilization assays. The interference of the EXP3174 ligand in the assay was assessed by measuring the signal inhibition exhibited by NPEXP (FIG. 9). The Michaelis Menten constant (Km) determined for both NPAng-I and NPEXPAng-I (FIG. 3E), resulted in values that were of the same order of magnitude as for the free ligand (cf. [1]) for both particle formulations. Additionally, the present inventors determined the catalysis constant (Kcat) to calculate the specificity constant (Kcat/Km) which is a useful indicator for comparing the affinity of different substrates for the same enzyme (FIG. 3F). The enzymatic activation of Ang-I on the NPEXPAng-I corona was not significantly different from the one on NPAng-I, indicating that ACE is not sterically hindered by the additional ligand EXP3174. Furthermore, the Kcat/Km value calculated based on the ligand concentration was equal for free and particle-bound Ang-I. More so, when Kcat/Km is calculated based on the NP concentration the bound ligand is a significantly better substrate for the enzyme, which is a result of the binding of several ligand molecules on the particle surface to several enzyme molecules (FIG. 3F).

### Example 4: Decision-Making NPs are Target-Cell Specific

All materials and methods mentioned in this example were as described in the previous examples.

After the particle interaction with their individual targets had been successfully established, the next step was to determine if NPs carrying an antagonist as well as an agonist on their corona would still trigger internalization by their target cells, and if so, if the uptake ensued from a specific ligand-receptor interaction. As antagonists do not cause AT1R-mediated endocytosis and agonists do, the present inventors investigated via confocal laser scanning microscopy (CLSM) the cellular localization of NPEXPAng-I in rMCs expressing YFP-tagged AT1R (pAT1R-rMCs). As shown in FIG. 4, NPEXPAng-I-associated fluorescence was found inside the cells. It increased with higher incubation times and strongly colocalized with the AT1R fluorescence.

Accordingly, there is a specific particle uptake, mediated by the AT1R. However, particles carrying only the antagonist (NPEXP) were not internalized by the cells and located mostly on the cellular surface (FIG. 10A). The particle fluorescence also colocalized with the receptor fluorescence, demonstrating a receptor-mediated attachment. As NPAng-I were also internalized by the cells (FIG. 10B), the enzymatically created Ang-II mediates the cellular uptake of NPEXPAng-I. Unexpectedly, there was a rearrangement of the receptors on the cellular membrane with increasing incubation times (FIG. 4), from a more diffuse and uniform cell membrane distribution (after 15 minutes) to a more concentrated clustering (at 90 minutes), which strongly colocalized with the NP fluorescence. This is additional evidence that the uptake is mediated by the AT1R, as the activation of receptors that are internalized via clathrin-coated pits, such as the GPCR like the AT1R, promotes receptor clustering.

For NPEXP a receptor rearrangement on the cell membrane also occurred, which is a result of a multivalent receptor binding promoted by receptor movement on the cellular surface. Once NPEXP attach to a receptor on the cell membrane, their lack of internalization can lead to receptor-particle mobility on the cell membrane, and further receptor binding. Particles without ligands (NPMeO) were not taken up by the cells (FIG. 10C), confirming that a specific targeting mechanism is essential to mediate a high cellular internalization.

Overall, the present inventors demonstrate that the presence of an attachment-mediating antagonistic ligand linked to the particle corona does not hinder subsequent particle internalization. More so, the inclusion of an additional ligand on the particle surface compensated the targeting loss due to stearic hindrance of the Ang-I ligand by the addition of a higher number of long polymer chains (FIG. 11). To further confirm the particle specificity and ligand-mediated internalization, the cells were pre-incubated for 30 minutes prior to particle addition with free EXP3174 or captopril, an ACE inhibitor, which resulted in a suppression of the particle-associated fluorescence analyzed by flow cytometry (FIG. 5A) and CLSM (FIG. 12).

Furthermore, the present inventors examined the particle internalization in different cell lines by flow cytometry (FIG. 5B). HeLa cells, which do not express ACE and only express minor AT1R levels, showed a low particle uptake, which was non-specific as it could not be suppressed by captopril or EXP3174. On the contrary, rMCs and HK-2 cells expressing both the targets were able to take up the particles, shown by the much higher particle-associated cell fluorescence. The internalization was also mediated by the activated proligand binding to the AT1R, as the preincubation of cells with captopril or EXP3174 significantly suppressed the cell fluorescence. Therefore, the particles show high specificity for their target cells. Nevertheless, when NPs enter the body, they are presented simultaneously with target and off-target cells. Therefore, the present inventors investigated if the NPEXPAng-I were able to differentiate between them.

Target cells (rMCs) were seeded together with an excess of off-target NCI-H295R or HeLa cells, which both lack the ACE and express high and low AT1R levels, respectively. They were incubated with the different NP formulations and each cell line was investigated for particle-associated fluorescence through flow cytometry (FIG. 5C-D). NPEXPAng-I showed outstanding target cell specificity, as they accumulated significantly more in target rMCs. The specificity is conferred by Ang-I as NPAng-I showed also low accumulation in both off-target cells. On the contrary, NPEXP bound to the cell surface to the same degree in rMCs as in NCI-H295R cells, which express high AT1R levels, demonstrating that a simple one-step recognition process is not enough to confer particle selectivity. CLSM images confirmed the flow cytometry findings (FIG. 5E and 13), where NPEXPAng-I- and NPAng-I fluorescence (red) was mostly associated with target rMCs (green) and not in off-target HeLa or NCI-H295R cells (white), while NPEXP fluorescence was found in both rMCs and AT1R-expressing NCI-H295R cells. Taken all together, these results demonstrate that the NPEXPAng-I uptake is receptor-mediated and that the initial cell attachment through the EXP3174 ligand does not reduce the particle specificity for the target cells conferred by the virus-mimetic recognition process.

### Example 5: NPs Target MCs In Vivo

All materials and methods mentioned in this example were as described in the previous examples.

Since the complementary targeting ability of both ligands on NPEXPAng-I and the particle specificity was demonstrated in vitro the next step was to determine whether the viral recognition principle would lead to a higher in vivo MC accumulation. To that end, targeted (NPEXPAng-I, NPAng-I and NPEXP) (FIG. 1A) and non-targeted (NPMeO) particle formulations were injected into NRMI mice and cryosections of the kidneys examined for particle-associated fluorescence (FIGS. 6 and 14A). As depicted in FIG. 6A, NPEXPAng-I fluorescence could be found homogeneously over all glomeruli in the kidney section, with no fluorescence in other kidney structures, such as the tubuli. On the contrary, for non-targeted NPMeO almost no NP fluorescence could be detected in the kidney sections (FIG. 6B). This demonstrates that simple size-dependent targeting is not enough to achieve a particle accumulation in MCs as NPMeO are probably cleared out of the mesangium due to their lack of specific cellular interaction. More so, NPEXP which are targeted NPs but not able to mediate cellular internalization also depicted very little glomerular fluorescence (FIG. 14A), demonstrating that particle uptake is fundamental to achieve a high MC accumulation. Furthermore, NPEXPAng-I achieved a much stronger and homogeneous glomerular distribution than NPAng-I, which lack the attachment factor (FIG. 14A), showing that in vivo an enhanced target cell recognition principle is highly advantageous.

In order to quantitatively assess the NP-associated fluorescence and better distinguish the differences among the different particle formulations, images of the glomeruli were taken at higher magnifications (FIG. 7A). Quantitative analysis of the glomerular fluorescence yielded a 15-fold higher fluorescence for virus-mimetic particles with enhanced recognition mechanism (NPEXPAng-I) compared to non-targeted control particles (NPMeO), which showed only small fluorescence spots in some glomeruli.

Additionally, NPEXPAng-I displayed significantly higher accumulation than one-ligand targeted particles (7- and 5-fold higher than NPEXP and NPAng-I, respectively) (FIG. 7B). That the detected florescence was particle-associated, was confirmed by the kidney distribution of the free dye used for particle labelling (CF647), which showed strong tubular but no glomerular fluorescence (FIG. 14B), as due to its small size it can be freely filtrated. To assess the NP glomerular distribution the fluorescence of the glomeruli in the outer and inner cortex was compared (FIG. 7C). For all particle formulations there were no significant differences among the two populations. This indicates that the particles are distributed homogeneously in the glomeruli of the entire kidney cortex, which is an indispensable prerequisite for the treatment of glomerular-associated diseases. Finally, as besides MCs there are other cells in the glomerulus which could have internalized the NPs, a specific antibody-staining of MCs using integrin-α8 as a marker was performed to ascertain that the particles accumulated in MCs. As depicted in FIG. 7D, the NPEXPAng-I fluorescence localized inside the antibody-stained MCs, confirming that the particles were able, not only to reach the glomerular mesangium, but also to be taken up by MCs.

Taken together these results clearly show that size-mediated targeting is a necessary prerequisite to reach the mesangium, but insufficient to achieve particle accumulation in MCs. NP internalization seems to be imperative to avoid mesangial clearance, which explains that particles lacking this trait (NPMeO and NPEXP) lead to the lowest glomerular fluorescence. Implementing a virus-mimetic recognition principle (NPAng-I) increases NP specificity and results in particle uptake which in turn leads to a higher MC-accumulation. However, facilitating the target cell recognition via an initial virus-like cell attachment (NPEXPAng-I) significantly enhances the NP's targeting potential, a result of a combined effect of the two ligands, as shown by the in vitro studies.

Furthermore, the enhanced functionalization of NPEXPAng-I does not lead to a decrease in the particle blood residence. Generally, NPs are coated with polymers such as PEG, which increase their circulation time and decrease plasma protein adsorption. A positive effect, which is usually counteracted by ligand functionalization, as off-target cells expressing the targeted receptors can bind and interfere with the NPs. Nevertheless, quantification of the plasma NP fluorescence one hour after injection showed that NPEXPAng-I remained in circulation to the same extent as non-targeted NPMeO and significantly longer than the other targeted formulations (FIG. 14C). This is probably due to a higher particle specificity resulting from a more complex cell recognition process. Overall, the results demonstrate that by closely mimicking the viral binding and internalization and combining it with an optimal NP size it is possible to develop NPs that target and massively accumulate in MCs.

### Example 6: Materials and Methods

Materials

Heterobifunctional hydroxyl poly(ethylene glycol)carboxylic acid with a molecular mass of 2000 and 5000 g mol⁻1 (COOH-PEG_{2k/5k}-OH) and hydroxyl poly(ethylene glycol)Boc-amine with a molecular mass of 2000 g mol⁻1 (Boc-NH-PEG₂ₖ-OH) were purchased from Jenkem Technology USA Inc. (Allen, Tex., USA) while methoxy poly(ethylene glycol)with a molecular mass of 5000 g mol⁻1 (MeO-PEG₅ₖ-OH) and Resomer RG 502 (PLGA) were obtained from Sigma-Aldrich (Taufkirchen, Germany). EXP3174 (also known as losartan carboxylic acid) was purchased from Santa Cruz (Heidelberg, Germany), while Cyclic RGDfK (cRGDfK) was obtained from Synpeptide Co. Ltd. (Shanghai. China). AlexaFluor™ 568 Hydrazide (Alexa568), CellTracker™ Green Dye (CTG) and CellTracker™ Deep Red Dye (CTDR) were purchased from Thermo Fisher Scientific (Schwerte, Germany). Amine-functionalized spherical gold NPs with an average diameter of 2.2 nm (Au_{2.2}-NH₂) were obtained from Nanopartz Inc. (Loveland, Colo., USA). GoldEnhance™ EM Plus kit was purchased from Nanoprobes (Yaphank, N.Y., USA). Goat-derived Integrin α-8 antibody was obtained from R&D Systems (Minneapolis, Minn., USA). All other chemicals were purchased from Sigma-Aldrich in analytical grade if not stated differently. Ultrapure water was obtained from a Milli-Q water purification system (Millipore, Billerica, Mass., USA). NCI-H295R (CRL-2128) and HeLa (CCL-2) cells were purchased from ATCC (Manassas, Va., USA). All cell lines were cultured in RPMI 1640 medium containing 10% fetal bovine serum, Insulin-Transferrin-Selenium (ITS) (1×) and 100 nM hydrocortisone.

Polymer Synthesis

COOH-PEG₂ₖ-PLA₁₀ₖ, Boc-NH-PEG₅ₖ-PLA₁₀ₖ and MeO-PEG₅ₖ-PLA₁₀ₖ block copolymers were synthesized via a ring-opening polymerization as previously described. In brief, heterobifunctional PEG polymers (1 equivalent=equiv) were mixed with 3,6-dimethyl-1,4-dioxane-2,5-dione (70 equiv) and 1,8-diazabicylo [5.4.0] undec-7-ene (3 equiv). The polymer mixture was stirred for 1 hour (h) at room temperature (RT) until polymerization was quenched with benzoic acid (14 equiv). Resulting block-copolymer was precipitated in diethyl ether, isolated via filtration and dried under vacuum. Molecular weight of synthesized polymers was determined in deuterated chloroform at 295 K using a Bruker Avance 300 spectrometer (Bruker BioSpin GmbH, Rheinstetten, Germany).

For preparation of cRGDfK-PEG₂ₖ-PLA polymers, previously synthesized COOH-PEG₂ₖ-PLA₁₀ₖ was covalently coupled to the lysine residue of cRGDfK as shown before. In short, COOH-PEG₂ₖ-PLA₁₀ₖ (1 equiv) was activated using 3-(ethyliminomethyleneamino)-N,N-dimethylpropan-1-amine (EDC)/N-hydroxysuccinimide (NHS) (25 equiv) for 2 h at RT, followed by quenching with β-mercaptoethanol (BME) (30 equiv). Activated polymer was reacted with cRGDfK (3 equiv) and N,N-diisopropylethylamine (DIPEA) (10 equiv) for 24 h at RT. After precipitation of resulting cRGDfK-coupled polymer in diethyl ether/methanol (15:1 V/V)), free cRGDfK and excess reactants were removed using dialysis against millipore water (mpH₂O).

For EXP3174-PEG₅ₖ-PLA₁₀ₖ, the Boc-protecting group of Boc-NH-PEG₅ₖ-PLA₁₀ₖ was initially cleaved. In brief, Boc-protected polymer was dissolved in dichloromethane (DCM)/trifluoroacetic acid (TFA) (1:1 V/V). After stirring for 30 minutes (min), excess TFA was neutralized using a saturated sodium hydrogen carbonate solution. The organic phase was washed with mpH₂O, followed by polymer isolation as described above. Resulting NH₂-PEG₅ₖ-PLA₁₀ₖ was coupled to EXP3174 via the carbonyl residue of the imidazole component. EXP3174 (3.5 equiv) was activated with N,N′-dicyclohexylcarbodiimide (DCC)/NHS (3.3 equiv) for 2 h at RT. After removal of resulting dicyclohexylurea via centrifugation, NH₂₋PEG₅ₖ-PLA₁₀ₖ (1 equiv) and DIPEA (17.5 equiv) were added and reacted for 24 h at RT. Resulting EXP3174-PEG₅ₖ-PLA₁₀ₖ was precipitated in methanol/diethyl ether (1:5 V/V) and the product was dialyzed against ethanol/100 mM borate buffer pH 8.₅/water (1/1/8 V/V) for 24 hours followed by mpH₂O for 12 h to remove unreacted EXP3174 and excess reactants.

PLGA Labeling with Fluorescent Dyes

For particle visualization, the core-forming PLGA was covalently linked to fluorescent dyes prior to NP preparation. To that end, carboxylic acid-terminated PLGA was activated for 2 h using 4-(4,6-dimethoxy-1,3,5-triazin-2-yl)-4-methylmorpholinium chloride (DMTMM) as a catalytical agent. Activated PLGA was then reacted either with AlexaFluor™568 Hydrazide or CFTM 647 amine for 24 h at RT. Labeled PLGA was dialyzed against mpH₂O for 24 h to remove unreacted fluorescent dyes.

PLGA Labeling with Nanogold

For electron microscopy analysis, PLGA was conjugated to nanogold. PLGA was initially activated with EDC and NHS for 2 h in DCM. After DCM removal under reduced pressure, activated PLGA was dissolved in DMSO and mixed with DIPEA and lyophilized monoamino gold nanoparticles with an average diameter of 2.2 nm (Au_{2.2}-NH₂). After stirring at RT for 24 h, gold-conjugated PLGA was precipitated in mpH₂O, isolated via centrifugation at 2500 g for 10 min and lyophilized.

NP Preparation

Block-copolymer nanoparticles were manufactured using a common solvent evaporation technique. Corresponding amounts of PEG-PLA polymers and PLGA were mixed at a ratio of 70/30 (m/m) and diluted in acetonitrile (ACN) to a final concentration of 10 mg mL⁻1. To reach the desired ligand surface density for hetero-/homo-functional NP species, cRGDfK-PEG₂ₖ-PLA and/or EXP3174-PEG₅ₖ-PLA₁₀ₖ were mixed with COOH-PEG₂ₖ-PLA₁₀ₖ according to the calibration depicted in FIG. 18d/e. The organic phase was then added dropwise to vigorously stirring 10% Dulbecco's Phosphate-Buffered Saline (DPBS) (7.5 mM, pH 7.4) and stirred for 3 h at RT to remove the organic solvent.

Resulting NP dispersions were concentrated via centrifugation at 1250 g for 25 min using Pall Microsep filters (molecular weight cut-off 30 kDa; Pall Corporation, N.Y., USA). To obtain the mass concentration of manufactured NPs, PEG content was assessed using a colorimetric iodine complexing assay. NPs were then lyophilized and gravimetrically analyzed to obtain the ratio of PEG content and NP weight. In the following experiments, this ratio was used to calculate mass concentration from the assessed PEG content for each NP species.

NP Characterization

NP size and ζ-Potential was evaluated using a Malvern Zetasizer Nano ZS (Malvern, Herrenberg, Germany). Samples were measured with a 633 nm He—Ne laser at an angle of 173° (25° C., RT) in 7.5 mM DPBS, using either PMAA semimicro cuvettes (DLS; Brand, Wertheim, Germany) or folded capillary cells (c-Potential; Malvern, Herrenberg, Germany).

cRGDfK Quantification

The level of cRGDfK on the NP surface was assessed based on the measurement of arginine. In brief, ₅₀ μL of NP samples (1 mg mL⁻1) were mixed with 175 μL of a working solution consisting of 9,10-phenanthrenequinone (150 μM in ethanol) and 2 N NaOH (6:1 V/V). After 3 h of incubation at 60° C., 1 equiv of sample was mixed with 1 equiv of 1 N HCl and incubated for another 1 h at RT. Finally, fluorescence was measured at a Synergy™ Neo2 Multi-Mode Microplate Reader (BioTek Instrument Inc., Winooski, Vt., USA) with an excitation wavelength of 312/7 nm and an emission wavelength of 395/7 nm. Dilutions of cRGDfK (0-40 μg mL⁻¹) served as calibration. cRGDfK molarity as well as the ratio of molar cRGDfK content and molar PEG content were determined and plotted against the theoretical value (FIG. 18d).

EXP3174 Quantification

To determine the surface level of EXP3174 on manufactured particles, 1 equiv of NP samples (1 mg mL⁻1) was mixed with 10 equiv of 0.2 M acetic acid. Dilutions of EXP3174 in 0.2 M acetic acid (0-30 μM) served as calibration. Fluorescence of samples and standards was measured at a Synergy™ Neo2 Multi-Mode Microplate Reader (see above) (excitation 250/10 nm, emission 370/5 nm). EXP3174 molarity as well as the ratio of molar EXP3174 content and molar PEG content was determined and plotted against the theoretical value (FIG. 18e).

Calcium Mobilization Assay

In order to investigate AT1r binding of NPs, intracellular calcium levels were measured using fura-2 as a Ca²⁺ chelator. In brief, rMCs were incubated with 5 μM fura-2AM, 2.5 mM probenecid and 0.05% Pluronics F-127 in Leibovitz's L-15 medium for 1 h at RT. Cells were thereafter centrifuged (5 min, 200 g, RT) and resuspended in Leibovitz's medium. 45 μL of NPs or free EXP3174 at different concentrations were pipetted into 96-well plates (Greiner Bio One, Frickenhausen, Germany), followed by 45 μL of rMC suspension (2×10⁶ mL⁻1). In the following, cells were incubated with samples for 45 min at RT. After incubation, 10 μL of 30 nM AT II was added to each well to activate uninhibited AT1r and consequently induce Ca²⁺ influx into the cell cytosol. Fluorescence signal during the first 30 seconds after injection was measured using a FluoStar Omega fluorescence microplate reader (BMG Labtech, Ortenberg, Germany) with excitation filters at 340/20 nm and 380/20 nm and the emission filter at 510/20 nm, respectively. Maximal ratio of Ca²⁺-bound to Ca²⁺-unbound Fura-2 was evaluated by incubating loaded cells with 0.1% Triton-X 100 and measuring fluorescence levels as described above. Analogously, minimal ratio was achieved by incubation with 0.1% Triton-X 100 combined with 45 mM ethylene glycol-bis(2-aminoethylether)-N,N,N′,N′-tetraacetic acid (EGTA). Levels of intracellular calcium per sample was calculated using the equation of Grynkiewicz et al. Half maximal inhibitory concentrations (IC₅₀) were calculated using GraphPad Prism (San Diego, Calif, USA) and applying a sigmoidal dose-response equation (variable slope).

CLSM Analysis

For detailed analysis of NP-cell interaction, rMCs were seeded into 8-well slides (Ibidi, Gräfelfing, Germany) at a density of 15.000 cells well⁻¹ and incubated for 24 hours at 37° C. In order to facilitate visualization of the cell cytosol, rMCs were stained with CTDR (25 μM, 45 min, 37° C.) in serum-free RPMI 1640 medium prior to seeding. NPs were manufactured using AlexaFluor™ 568-labeled PLGA and adjusted to 0.05 mg mL⁻1 in Leibovitz's buffer supplemented with 0.1% BSA. Mesangial cells were incubated with 250 μL of NPs for 15, 45 and ₉0 min at 37° C., washed with prewarmed DPBS and fixed with 4% paraformaldehyde (PFA) in DPBS for 10 min. After a final washing step, fixed samples were analyzed using a Zeiss LSM 710 (Carl Zeiss Microscopy GmbH, Jena, Germany).

Flow Cytometry

To assess mesangial cell association of NP samples, rMCs were seeded into 24-well plates (Greiner Bio One, Frickenhausen, Germany) at a density of 40.000 cells well⁻¹ and incubated for 48 h at 37° C. NPs were manufactured using CF™ 647-labeled PLGA and adjusted to 0.05 mg NP mL⁻¹ in Leibovitz's buffer supplemented with 0.1% bovine serum albumine (BSA). To confirm α_{V}β₃-dependence of NP cell entry, 300 μL of free cRGDfK (c=500 μM) were added to the relevant cell samples for 15 minutes prior to NP incubation. Cells were washed with DPBS and 300 μL of prewarmed NP solutions were added for 60 minutes at 37° C. For respective analysis of time-dependent uptake, cells were incubated over a time period of 120 minutes with NPs being removed after 0, 15, 30, 45, 60, 90 and 120 minutes. Cells were washed with DPBS, trypsinized and centrifuged for 5 min at 200 g and 4° C., followed by two further washing and centrifugation steps (DPBS, 200 g, 5 min, 4° C.). Final samples were resuspended in DPBS and analyzed using a FACS Calibur cytometer (Becton Dickinson, Franklin Lakes, N.J., USA). NP-associated fluorescence was excited at 633 nm and corresponding emission was recorded (661/16 bandpass filter). Flow cytometry data was analyzed using Flowing software 2.5.1 (Turku Centre for Biotechnology, Turku, Finland). Within the population of viable cells, geometric mean of cell-associated fluorescence was evaluated.

Transmission Electron Microscopy

To evaluate cellular localization of NPs, rMCs were seeded into a 24-well plate at a density of 12.000 cells well⁻¹ and incubated for 72 hours. NP formulations containing nanogold-conjugated PLGA were diluted in Leibovitz's buffer containing 0.1% BSA and added for 45 min at a concentration of 0.05 mg mL⁻¹ (V=300 μL). After incubation, samples were washed with DPBS and prepared for electron microscopy analysis. In brief, cells were fixed with 2.5% PFA and 2.5% glutaraldehyde in a 0.1 M sodium cacodylate solution (Caco buffer) for 60 min at RT, washed with Caco buffer and permeabilized with 0.1% Triton-X in DPBS for 10 min. After a washing step with mpH₂O, samples were gold enhanced using a GoldEnhance™EM Plus kit (Nanoprobes Inc., Yaphank, N.Y., USA) according to the manufacturer's specifications, followed by further washing and post-fixation in a 2.5% sodium thiosulfate solution in mpH₂O. Cells were stained with 0.5% osmium tetroxide and dehydrated in rising concentrations of ethanol (50-99.5%) For counterstaining, 2% uranylacetate was applied for 5 min at 70% ethanol concentration. After embedding in Epon, ultrathin sections of 150 nm were imaged using a 100 kV Zeiss Libra 120 electron microscope (Carl Zeiss NTS GmbH, Oberkochen, Germany) at a magnification of 6300× as well as 12500×.

Co-Culture Experiments

In order to assess cell selectivity of manufactured NPs, the present inventors used a co-culture design that had been previously implemented by the inventors. For flow cytometry analysis, rMCs were seeded together with HeLa or NCI-H295R cells in 24-well plates at a density of 10.000 and 75.000 cells well⁻¹ respectively and incubated for 48 h at 37° C. To differentiate between cell types, rMCs were stained with CTG (15 μM, 45 min, 37° C.) in serum-free RPMI 1640 medium prior to seeding. Co-cultured cells were then incubated with CF™647-labeled NPs at a concentration of 0.05 mg mL⁻¹ (V=300 μL) for 45 min. Preparation of samples and flow cytometry analysis was performed as described above. Additionally, rMC-associated fluorescence was excited at 488 nm and recorded using a 530/30 bandpass filter. During data analysis, the population of viable cells was further gated for stained rMC cells and NP-associated fluorescence analyzed concerning cell specificity. For CLSM analysis, rMC cells were CTG-stained prior to seeding as described above. To visualize all cell types, HeLa or NCI-H295R cells were also stained using CTDR (25 μM, 45 min, 37° C.). After CellTracker™ incubation, rMCs were seeded into 8-well Ibidi slides together with HeLa/NCI-H295R cells at a density of 2.000 and 10.000/20.000 cells well⁻¹. After 48 h of incubation at 37° C., cell nuclei were stained with Hoechst 33258 (5 μg mL⁻¹ in DPBS) for 20 minutes. Cells were washed twice with prewarmed DPBS and AlexaFluor™568-labeled NPs were added at a concentration of 0.05 mg mL⁻¹ (V=250 μL) for 45 min at 37° C. After NP incubation, samples were treated as described above and analyzed using a Zeiss LSM 710 microscope.

In Vivo Cell Targeting

Animal experiments were performed according to the national and institutional guidelines and were approved by the local authority (Regierung von Unterfranken, reference number: 55.2-2532-2-329). Female, 10-week-old NMRI mice (Charles River, Sulzfeld, Germany) acted as model animals. After analgesia with buprenorphin (0.1 mg kg body weight⁻¹), mice were anaesthetized with 2.5% isoflurane and 100 μL of CF™ 647-labeled NPs (c=120 nM) were injected via the vena jugularis. Mice were kept in anesthesia and after 5 min, an initial blood sample was taken via i.v. punction. After 60 minutes, mice were anaesthetized with ketamine/xylazine, a final blood sample was taken, and animals were killed via perfusional fixation. Both kidneys were removed and immediately transferred to a 18% sucrose and 14% PFA solution in phosphate buffer (0.1 M pH 7.4). After 6 h, kidneys were washed with DPBS and cryoprotected at −80° C. until further processing. For cryosections, the organs were embedded in Tissue Tek® O.C.T.™ Compound (Sakura Finetek, Torrance, Calif., USA), cut into 5 μm sections using a CryStar NX70 cryotome (Thermo Fisher Scientific, Waltham, Mass., USA) and fixed on Superfrost198 plus glass slides (Thermo Fisher Scientific, Schwerte, Germany). For analysis of NP kidney deposition and glomerular fluorescence quantification, sections were rinsed in DPBS and blocked with 5% BSA supplemented with 0.04% Triton-X in DPBS for 10 min at RT. After further rinsing in DPBS, samples were stained for cell nuclei with a 1:400 dilution of 4′,6-Diamidine-2′-phenylindole dihydrochloride (DAPI) in 0.5% BSA and 0.04% Triton-X in DPBS. After a final washing step in DPBS and mpH₂O respectively, cryosections were mounted with Mowiol mounting medium and analyzed at a Zeiss Axiovert 200M. For image analysis, Fiji software (Madison, Wis., USA) was used. Glomerular fluorescence intensities were evaluated by measuring the integrated density of areas over a certain fluorescence threshold and division by the glomerular area. In order to assess the exact cellular location of NPs, kidney cryosections were prepared as described above. After washing and blocking of sections, samples were stained overnight at 4° C. with a goat-derived Integrin α-8 antibody (1:200 dilution in 0.5% BSA/0.04% Triton-X in DPBS). Samples were thereafter washed with DPBS and stained with a 1:400 dilution of Cyt® donkey anti-goat and DAPI in 0.5% BSA/0.04% Triton-X in DPBS for ih at RT. After a final washing step, samples were mounted and analyzed at a Zeiss LSM 710.

### Example 7: Preparation of Hetero-Multivalent EXPcRGD NPs Using a Modular Concept

All materials and methods mentioned in this example were as described in example 6.

In order to create NPs with the desired adenovirus-mimetic properties, the present inventors implemented a modular design that is based on the synergistic combination of different biocompatible polymer components into a hetero-multivalent particle species (FIG. 18a). The overall polymer composition of the NPs was intended to be similar to the present inventors' previous Influenza A mimetic NP design in order to be able to adequately compare both targeting concepts. Thereby, widely established poly(lactic-coglycolic acid) (PLGA) forms a hydrophobic NP core that not only guarantees enhanced structural integrity in aqueous media but also allows NP visualization via coupling of fluorescent dyes or nanogold. Poly(ethylene glycol)-poly(lactic acid) (PEG-PLA) block copolymers as second component offer the structural flexibility that is needed in order to implement the pursued virus-mimetic NP design. In a first step, PEG-PLA polymers with either longer (PEG₅ₖ-PLA₁₀ₖ ) or shorter (PEG₂ₖ-PLA₁₀ₖ ) PEG chains were synthesized via a previously described ring-opening polymerization (FIG. 25a). Since EXP3174 was intended to initially bind the mesangial AT1r as a freely moving ligand, it was covalently coupled to the longer and thus more flexible PEG₅ₖ-PLA₁₀ₖ chains (FIG. 25b). The second ligand (cRGDfK), in contrast, should not be able to interact with surface-bound integrins unless a first AT1r binding and subsequent spatial approach of the NP has taken place. To that regard, it was attached to shorter PEG₂ₖ-PLA₁₀ₖ (FIG. 25c). Surface density of both cRGDfK and EXP3174 could be tuned precisely by mixing distinct amounts of either ligand-functionalized or unfunctionalized PEG-PLA polymers with PLGA prior to NP manufacture via nanoprecipitation (FIG. 18d/e).

The present inventors decided to prepare hetero-functional nanoparticles that carry 25% EXP3174 and 15% cRGDfK on their surface (EXPcRGD NPs), thereby sufficiently exploiting the ligands' receptor binding capacities but preserving structural integrity of manufactured particles. Particles should be able to locate the target cell by binding to the AT1r via sterically flexible EXP3174, then lower the spatial distance to the cell surface and subsequently activate αVβ3 integrins via previously concealed cRGDfK, which eventually initiates NP endocytosis (FIG. 17c). Hetero-functional EXPcRGD NPs as well as homo-functional (EXP NPs/cRGD NPs) and non-functionalized, methoxy-terminated particles (Control NPs) were manufactured below a size threshold of 60 nm and showed negative zeta potential values (FIG. 18b/c). These characteristics should not only facilitate successful extravasation through endothelial fenestrations (FIG. 17 a/b), but also prevent NP phagocytosis or extended serum protein adsorption.

### Example 8: Hetero-Multivalent EXPcRGD NPs Display Excellent Ligand Affinity for Target Motifs

All materials and methods mentioned in this example were as described in example 6 and 7.

The present inventors tested EXP3174-mediated NP binding to the AT1r expressed by rat mesangial cells (rMCs). As activation of G_{q}-coupled AT1r with its primary ligand angiotensin II (AT II) results in a calcium influx into the cell cytosol, intracellular Ca²⁺ levels after AT II stimulation can be used as a marker for AT1r activity after NP incubation. Thereby, low receptor activity indicates a high ratio of bound EXP3174, as the ligand itself acts as a potent antagonist. FIG. 19 shows intracellular Ca²⁺ levels of AT II-stimulated rMCs that had been pre-incubated with NPs or free EXP3174 for 45 minutes. Both EXPcRGD NPs (IC₅₀=276±31 pM) and EXP NPs (IC₅₀=552±73 pM) showed excellent AT1r avidity, resulting in a highly effective inhibition of the receptor in the picomolar range and consequent minimal intracytosolic Ca²⁺ levels (FIG. 19a). Furthermore, inhibition potency of EXP3174-carrying NP types was even higher than for the free ligand (IC₅₀=2.66±0.9 nM). This strongly suggests that EXP3174-functionalized particles were able to interact with the target receptor in a multivalent fashion, leading to an overall avidity gain. As IC₅₀ levels of both EXPcRGD and EXP NPs were found to be in the same range, the present inventors concluded that the combination of both EXP3174 and cRGDfK in one particle type did not significantly interfere with the binding capacity of EXP3174 itself. Moreover, Control NPs and cRGD NPs did not show any interaction with the AT1r, resulting in a maximal Ca²⁺ signal upon receptor stimulation and confirming the assay's specificity for the AT1r (FIG. 19b).

Having verified the AT1r binding capacity of adenovirus-mimetic EXPcRGD NPs, the next step was to investigate particle uptake into rMCs via cRGDfK-αVβ3 interaction. The present inventors therefore incubated mesangial cells with fluorescently labeled NPs and analyzed the cellular distribution via confocal laser scanning microscopy (CLSM). In order to visualize the cell body, rMCs were pre-treated with CellTracker™ Deep Red (CTDR). FIG. 20a shows strong intracellular accumulation of AlexaFluor™568-labeled EXPcRGD NPs in spherical structures that represent endocytotic vesicles. Over time, both the number and intensity of visible accumulations increased. Additionally, vesicles appeared to gain size with longer incubation times. These findings support that cRGDfK-functionalized NPs are able to bind the target cell and be taken up into intracellular vesicles via integrin-mediated endocytosis. Over time, these endocytotic vesicles fuse to larger endosomes and therefore gain size as well as intensity.

Building on CLSM results, the present inventors performed flow cytometry analysis of NP-treated rMCs and determined the cell-associated fluorescence over an incubation period of 120 minutes. As shown in FIG. 20b, levels of NP-derived fluorescence were maximal for EXPcRGD NPs compared to all other NP species over the entire incubation period. While EXP NPs as well as Control NPs merely showed moderate fluorescence signals, substantial levels of cell-association could be detected for cRGD NPs. Remarkably, respective fluorescence levels reached a plateau after approximately 60 minutes, while EXPcRGD NPs' cell-association further increased. This strongly supports the hypothesis of a sequential interaction between EXPcRGD NPs and their target cell which results in a prolonged increase of fluorescence levels compared to homo-functional cRGD NPs.

To investigate the impact of αVβ3 integrin on EXPcRGD NP cell uptake, an excess of free cRGDfK (c=500 μM) was added prior to incubating rMCs with EXPcRGD NPs for 60 min. As a result, levels of cell-associated fluorescence sharply decreased to levels comparable with those of EXP NPs or Control NPs (FIG. 20c). Hetero-multivalent particles apparently were no longer able to address the necessary αVβ3 integrin and therefore could not substantially initiate endocytosis after AT1r binding.

To further verify the concept of integrin-mediated NP endocytosis, the present inventors decided to utilize transmission electron microscopy (TEM), which enabled the assessment of NP-cell interactions at a much higher magnification level. In order to increase electron density and consequential TEM visibility of applied NPs, ultrasmall gold nanoparticles with an average diameter of 2.2 nm were covalently coupled to PLGA that was then used for further NP manufacture (FIG. 26). Mesangial cells that were incubated with these gold-tagged NPs could then be gold-enhanced in order to intensify and thus visualize the particles' gold core and assess their exact location. This retrospective gold-enhancement offers the substantial advantage that physicochemical characteristics of nanogold-labeled NPs do not significantly differ from unlabeled NPs, which would not be the case for usually utilized gold NPs.

FIG. 21a shows the cell body of two mesangial cells that had been incubated with gold-tagged EXPcRGD NPs. Within the cell cytosol, numerous circular vesicles, filled with gold-enhanced NPs, could be detected. The distribution pattern showed remarkable similarity to the previously described CLSM results (FIG. 20a), thereby strongly supporting ligand-mediated NP endocytosis. Additionally, particles were observed to have accumulated at the cell border, indicating that these NPs were still bound to membrane-located surface structures. These findings further indicate that applied EXPcRGD NPs interacted with the target cell in a stepwise process of prior binding to the AT1r and subsequent integrin-mediated endocytosis. In accordance with this assessment, EXP NPs without surface-bound cRGDfK could only be detected at the rMC membrane while no particle accumulations in endocytotic vesicles were found (FIG. 21b). Additionally, cell-particle association for Control NPs was only marginal (FIG. 21c). Specificity of the applied gold enhancement was shown by a lack of gold accumulation in particle-free cells.

### Example 9: Ligand Synergism Leads to an Enhanced Mesangial Cell Selectivity In Vitro

All materials and methods mentioned in this example were as described in examples 6 to 8.

Having demonstrated that hetero-multivalent EXPcRGD NPs synergistically combine both key features of its surface ligands and present them in a sterically controlled manner, the present inventors intended to demonstrate that this design can actually be utilized to increase mesangial cell selectivity. The present inventors therefore implemented an in vitro based assay, in which target rMCs were co-cultured with a superior number (5-10 fold) of off-target cells carrying none or merely one of the two target receptors. While HeLa cells expressed neither AT1r nor αVβ3-integrin to a significant degree, NCI-H295R cells were chosen as they showed a high AT1r but low αVβ3 expression (FIG. 27).

To differentiate between co-cultured cells in CLSM analysis, CellTracker™ Green (CTG) was used to stain rMCs while off-target cells were marked with CTDR. After 45 minutes of incubation with fluorescently labeled EXPcRGD NPs, cellular distribution of NPs was assessed. In the rMC/HeLa co-culture model, particle-derived fluorescence could almost exclusively be detected within the areas of mesangial cells. HeLa cells, in contrast, showed merely weak interaction with NPs, resulting in marginal fluorescence levels (FIG. 22a). The present inventors accordingly concluded that EXPcRGD NPs could selectively locate mesangial cells among HeLa cells due to the differences in receptor expression on the cell surface. These findings were supported by flow cytometry analysis showing that cell-associated fluorescence of EXPcRGD NPs was significantly higher in rMCs than in off-target HeLa cells, while cell interaction for Control NPs was only marginal for both cell types (FIG. 22b). In contrast, rMC/NCI-H295R co-culture provided a divergent particle distribution. NP-associated fluorescence could not only be found in rMCs, but also in areas covered by NCI-H295R cells. However, distribution patterns differed significantly. While fluorescence among rMCs was found in circular, vesicle-like structures as seen before, NCI-H295R-associated fluorescence was more diffuse and intensified mainly at the cell membrane (FIG. 22a). The present inventors therefore concluded that, while accumulating in endocytotic vesicles of rMCs, EXPcRGD NPs were merely able to bind AT1r present in the cell membrane of NCI-H295R cells but could not be taken up into the cytosol due to an absence of αVβ3 integrin. Additionally, flow cytometry analysis showed that even though NP-associated fluorescence for NCI-H295R cells was higher compared to HeLa cells, EXPcRGD NPs still showed a significantly enhanced signal in mesangial cells (FIG. 22c). Remarkably, addition of an excess of free EXP3174 (c=1 mM) prior to NP incubation led to a sharp decrease of fluorescence levels for NCI-H295R cells, while cell-associated fluorescence for rMCs was still significantly higher. This observation further supports the notion that EXPcRGD NPs are actually able to utilize both surface ligands to target mesangial cells, thereby profiting from the hetero-functional design.

In summary, the present inventors' co-culture model demonstrated that hetero-multivalent EXPcRGD NPs have the capability to effectively identify receptor-positive mesangial cells in the presence of off-target cells that are not only prevailing in number but even express one of the two target receptors.

### Example 10: Accumulation of Adenovirus-Mimetic EXPcRGD NPs in Mesangial Cells In Vivo

All materials and methods mentioned in this example were as described in examples 6 to 9.

Both rMC binding and uptake studies successfully showed that the present inventors' virus-mimetic concept of sequential ligand-receptor interaction enables hetero-multivalent EXPcRGD NPs to selectively target mesangial cells in vitro. However, transferring in vitro results into a robust system with sufficient in vivo efficiency has been shown to be the major obstacle in nanoparticle design as many strategies fail to deliver desired target-specificity. The present inventors therefore decided to assess the NPs' capability to actually reach mesangial areas in vivo which does not only require active cell uptake facilitation but also adequate passive accumulation in the target region. To that regard, fluorescently labeled NPs were injected into io-week-old female NMRI mice. After 1 h of NP circulation, mice were sacrificed, and kidneys were extracted. Fluorescence analysis of prepared cryosections revealed that EXPcRGD NPs effectively accumulated in glomerular areas while fluorescence in tubular parts of the kidney was neglectable (FIG. 23). On the contrary, Control NPs as well as homo-functional EXP or cRGD NPs showed a considerably lower deposition in kidney cryosections.

To quantify observed differences, glomerulus-associated fluorescence levels were determined by assessing the glomerular fluorescence intensity per area for all NP types (FIG. 24a/b). EXPcRGD NPs thereby showed a more than io-fold increase in fluorescence intensity compared to control NPs. Moreover, glomerular accumulation of hetero-multivalent NPs was significantly greater than for both homo-functional NP types. Remarkably, cRGD NP fluorescence was even lower than for non-functionalized Control NPs. The present inventors hypothesize that cRGD NPs were not able to reach glomerular areas as a predominant number of particles bound αVβ3-expressing endothelial cells shortly after injection and consequently left the bloodstream before reaching deeper areas of the kidney. This hypothesis was further supported by the finding that relative blood plasma levels of cRGD NPs after ih of incubation was minimal among all particle types (FIG. 28a). In EXPcRGD NPs, on the contrary, shorter cRGDfK-functionalized PEG-PLA chains were shielded from premature exposition to αVβ3 integrins by addition of longer, EXP3174-functionalized PEG-PLA chains. Consequently, hetero-multivalent particles avoided off-target deposition and therefore successfully reached glomerular areas within the kidney. Antibody staining for mesangial cell marker integrin-α8 further revealed that EXPcRGD NP-associated fluorescence in the glomerulus could almost entirely be found within mesangial cells (FIG. 24c), proving the hypothesis of extravasation into the mesangial interstitium and subsequent endocytosis (FIG. 17a/b). In order to verify that detected fluorescence in mesangial areas was derived from structurally intact NPs, the present inventors additionally injected a comparable dose of free fluorescent dye into mice and analyzed fluorescence deposition. While intraglomerular signal for these samples was negligible, tubular cells exhibited very strong fluorescence levels (FIG. 28b). This indicated that, in contrast to injected NP species, the low-molecular dye was renally filtrated. The present inventors therefore concluded that intraglomerular fluorescence for all NP types derived from intact particles as a degradation would have led to an increase in the tubular signal, otherwise.

Thus, the herein discussed in vivo studies successfully demonstrated the potential of the new, adenovirus-mimetic NP design of the present invention. Hetero-multivalent EXPcRGD NPs effectively accumulated in mesangial areas of the glomerulus while homo-functional or unfunctionalized NP species failed to do so. This strongly suggests that in order to reach sufficient levels of bioavailability, NPs do not only have to carry appropriate surface ligands but must also present them in an orchestrated fashion that is suitable for the respective targeting strategy. Moreover, NP accumulation in the mesangium also proved that the adenovirus-mimetic system of sterically controlled particle-cell interaction is highly effective.

Example 11: Cinaciguat-Loaded EXPcRGD NPs Show High Efficiency

Nanoparticles (NPs) using either an influenza A mimetic or adenovirus mimetic target cell recognition concept were detected to be efficiently accumulating within mesangial cells in an in vivo setting. In a next step, the experimental drug cinaciguat (BAY 58-2667) was encapsulated in adenovirus-mimetic EXPcRGD NPs. Cinaciguat (CCG) is a potent activator of the soluble guanylate cyclase (sGC) and has been shown to significantly decrease mesangial fibrosis and reduce glomerular damage in a diabetes animal model. By encapsulating CCG in the inventors' promising NP species, cell-selective delivery of CCG to pathological mesangial sites can be considerably increased, leading to an enhanced therapeutic effect with minimized off-target effects (FIG. 29).

In the inventors' experimental set-up, CCG was initially encapsulated in hetero-multivalent EXPcRGD NPs. Resulting NPs carried approximately 500-700 CCG molecules per NP (data not shown). In all following experiments, the administration of free cinaciguat at a concentration of 2 μM was compared to CCG-loaded EXPcRGD NPs at a concentration of approximately 0.5 nM (equaling 0.2 μM of CCG) and drug-free EXPcRGD control NPs (FIG. 30). The concentration of free cinaciguat was chosen in accordance with previous publications that showed an anti-fibrotic effect of CCG in this concentration range. CCG-carrying NPs however carried only 10% of respective CCG amount to test a possible drug delivery effect.

To assess the effect of CCG-loaded EXPcRGD NPs on target sGC, mesangial cells were initially incubated for 24 h and protein quantity was assessed using Western Blot analysis. Interestingly, the overall amount of sGC was thereby gradually increasing both after incubation with free drug and CCG-loaded NPs, indicating not only the previously shown activating but also a stabilizing effect of cinaciguat on the sGC (FIG. 31). In contrast, NPs lacking cinaciguat did not show any significant effect on sGC levels.

Finally, the anti-fibrotic and anti-proliferative potential of a NP-assisted CCG delivery was analyzed. In that regard, mesangial cells were initially incubated for 4 h either with free CCG, CCG-loaded EXPcRGD NPs or control NPs without encapsulated drug. After 4 h, 10 ng mL⁻¹ of transforming growth factor β (TGF-β) were added for 48 h to induce a fibrotic and hyperproliferative remodeling. While administration of TGF-β lead to a considerable increase in mesangial cell proliferation, pre-incubation with both free CCG and CCG-loaded NPs could significantly reverse this effect (FIG. 32). Also, Western Blot and fluorescence microscopy analysis of fibrosis markers α-SMA and collagen I revealed a similar effect of CCG(-loaded NPs) on inhibiting pro-fibrotic remodeling of mesangial cells.

Taken together, these results revealed two major outcomes:

1. Both free and NP-encapsulated cinaciguat showed a significant effect on its target enzyme sGC, leading to a considerable activation of described anti-fibrotic pathway (FIG. 29). These results were in line with previous findings on CCG and show the remarkable potential of the therapeutic agent in an anti-fibrotic therapy.

2. Throughout all experiments, cinaciguat-loaded EXPcRGD NPs showed effects that were comparable to the administration of free CCG even though the overall amount of encapsulated CCG was only 10% of the free drug dose (0.2 μM vs. 2 μM). This indicates the considerable potential of described NPs to more efficiently deliver the pharmaceutical agent to its intended intracellular target.

### REFERENCES

[1] Maslanka Figueroa, S., Veser, A., Abstiens, K., Fleischmann, D., Beck, S., and Goepferich, A. (2019). Influenza A virus mimetic nanoparticles trigger selective cell uptake. Proc. Natl. Acad. Sci. 201902563.

[2] Sah E. and Sah H. Journal of Nanomaterials, Volume 2015, Article ID 794601.

[3] Hennig, R., Ohlmann, A., Staffel, J., Pollinger, K., Haunberger, A., Breunig, M., Schweda, F., Tamm, E. R., and Goepferich, A. (2015). Multivalent nanoparticles bind the retinal and choroidal vasculature. J. Control. Release 220, 265-274.

[4] Inuzuka, T., Fujioka, Y., Tsuda, M., Fujioka, M., Satoh, A. O., Horiuchi, K., Nishide, S., Nanbo, A., Tanaka, S., and Ohba, Y. (2016). Attenuation of ligand-induced activation of angiotensin II type 1 receptor signaling by the type 2 receptor via protein kinase C. Sci. Rep. 6, 21613.

The features of the present invention disclosed in the specification, the claims, and/or in the accompanying figures may, both separately and in any combination thereof, be material for realizing the invention in various forms thereof

1-17. (canceled)

18. A nanoparticle, comprising a nanomaterial and at least a first ligand and a second ligand,
wherein said first ligand is capable of mediating an attachment of said nanoparticle to a target cell, and
wherein said second ligand is capable of mediating an internalization of said nanoparticle into said target cell.

19. The nanoparticle according to claim 18, wherein said first ligand is a non-agonistic agent binding to a GPCR, and/or an agent binding to glycoprotein and/or glycolipid on a target cell surface.

20. The nanoparticle according to claim 18, wherein said second ligand is any of i) an agent binding to an integrin, ii) an agonistic agent binding to a GPCR, iii) an agent binding to an ectoenzyme, and/or iv) an agent binding to a transferrin-receptor.

21. The nanoparticle according to claim 18, further comprising a therapeutic agent.

22. The nanoparticle according to claim 18, wherein said first ligand and said second ligand are each coupled to said nanomaterial.

23. The nanoparticle according to claim 18, wherein said nanomaterial comprises more than one block-copolymer chain, and wherein said first ligand is coupled to a first block-copolymer chain of said nanomaterial and said second ligand is coupled to a second block-copolymer chain of said nanomaterial, and wherein said first block-copolymer chain is longer than said second block-copolymer chain.

24. The nanoparticle according to claim 23, wherein said first block-copolymer chain comprises PEG in a range of from 1 k to 20 k, and/or comprises PLA in a range of from 5 k to 40 k.

25. The nanoparticle according to claim 18, wherein said second ligand is enzymatically activated prior to said internalization of said nanoparticle into said target cell.

26. The nanoparticle according to claim 18, wherein said target cell is selected from a mesangial cell, an endothelial cell, a B cell, a T cell, a macrophage, a dendritic cell, and a tumor cell.

27. The nanoparticle according to claim 18, wherein said nanoparticle has a size of from 5 nm to 1000 nm.

28. The nanoparticle according to claim 18, wherein a ratio of said first ligand to said second ligand is in the range of from 2:1 to 1:2.

29. The nanoparticle according to claim 18, wherein said nanoparticle has a particle avidity for a targeted receptor of from 1 pM to 100 nM.

30. The nanoparticle according to claim 18, wherein said nanomaterial comprises PEG and wherein said nanoparticle has a ligand density of ligand/PEG of at least 5%.

31. A medicament or a diagnostic agent, comprising a nanoparticle as defined in claim 18.

32. A method of preventing or treating a disease selected from diabetic nephropathy, glomerulonephritis, glomerular VEGF A dysregulation, endothelial VEGF A dysregulation, diabetic retinopathy, rheumatoid arthritis, age-related macular degeneration, and cancer, wherein said method comprises administering an effective amount of a nanoparticle, as defined in claim 18, to a patient in need thereof.

33. A method of preparing a nanoparticle, as defined in claim 18, comprising the steps:
a) providing, in any order, one or several nanomaterial(s) and, optionally, a therapeutic agent;
b) optionally, preparing a block-copolymer from any of said one or several nanomaterial(s);
c) coupling, in one or more steps, a first ligand and a second ligand thereto;
d) providing a therapeutic agent, if not already provided in step a);
e) preparing and obtaining a nanoparticle using the ligands coupled to said nanomaterial and said therapeutic agent.

34. The method according to claim 34, wherein said obtaining in step e) comprises obtaining nanoparticles having a polydispersity index of from 0.01 to 0.5.

35. The nanoparticle according to claim 18, wherein said nanomaterial comprises any of polyethylene glycol (PEG), polylactic acid (PLA), poly(lactic-co-glycolic acid) (PLGA), an oxazoline-derived polymer, a poly(aminoacid), a polysaccharide, a phospholipid, a sphingolipid, cholesterol, a PEG-lipid, a block-copolymer, gold, or a qdot material.

36. The nanoparticle according to claim 18, wherein said first ligand is angiotensin II receptor type 1 (AT1r), human neuropeptide Y1-receptor, C-X-C chemokine receptor type 4, heparan sulfate, a sialoglycoprotein, a ganglioside, or a mannose receptor.

37. The nanoparticle according to claim 18, wherein said second ligand is αVβ3 integrin, αVβ5 integrin, AT1r, legumain, a membrane-type matrix metalloproteinase, angiotensin converting enzyme (ACE), or an agent binding to a transferrin-receptor.
